(19) United States
(12) Patent Application Publication
(10) Pub. No.: US 2023/0218723 A1
(43) Pub. Date: Jul. 13, 2023

(54) COMPOSITIONS AND METHODS FOR USING SILK-ELASTINLIKE PROTEIN-BASED POLYMERS

(71) Applicant: University of Utah Research Foundation, Salt Lake City, UT (US)

(72) Inventors: Hamidreza Ghandehari, Salt Lake City, UT (US); Joseph Cappello, San Diego, CA (US); Azadeh Poursaid, Salt Lake City, UT (US); Mark Martin Jensen, Pleasant Grove, UT (US)

(21) Appl. No.: 17/640,627

(22) PCT Filed: Sep. 4, 2020
(86) PCT No.: PCT/US2020/049458

§ 371 Date: Mar. 4, 2022

Related U.S. Application Data
(60) Provisional application No. 62/897,033, filed on Sep. 6, 2019.

(51) Int. Cl. A61K 38/39; A61K 9/00; A61K 49/00; A61P 9/14

(52) U.S. Cl. CPC A61K 38/39; A61K 9/0024; A61K 49/0034; A61K 49/0073; A61P 9/14

(57) ABSTRACT

Disclosed are methods of treating an aneurysm in a subject comprising administering to the subject a therapeutically effective amount of a composition comprising a SELP. Disclosed are methods of preventing rupture of an aneurysm comprising administering to a subject having an aneurysm a composition comprising a SELP, wherein the SELP is present in the aneurysm and prevents rupture. Also disclosed are methods of embolizing an aneurysm in a subject comprising administering to the subject a therapeutically effective amount of a composition comprising SEEP. Disclosed are methods of treating AVM in a subject comprising administering to the subject a composition comprising a SELP. In some aspects, the SELP embolizes an abnormal blood vessel in the AVM. Disclosed are methods of embolizing an AVM in a subject comprising administering to the subject a therapeutically effective amount of a composition comprising a SELP, wherein the SELP embolizes an abnormal blood vessel in the AVM.

### CROSS-REFERENCE TO RELATED APPLICATION

This application claims the benefit of U.S. Provisional Patent Application No. 62/897,033, filed on Sep. 6, 2019, which is incorporated by reference herein in its entirety.

### STATEMENT REGARDING FEDERALLY SPONSORED RESEARCH

This invention was made with government support under Grant Number 1R41NS100184 awarded by the National Institute of Health and Grant Number 1256065 awarded by the National Science Foundation. The government has certain rights in this invention.

### BACKGROUND

Cerebral aneurysms (CA), bulges in weakened blood vessels in the brain, are the primary cause of severe hemorrhagic stroke. Current embolic systems for treating CA leave behind metal components permanently in the brain that interfere with medical imaging, require the use of specialized equipment, fail to resolve the aneurysm in up to 40% of patients, and can increase the risk of death in the event of aneurysm rupture. An ideal embolic system for treating CA would be easily deployed with any clinical microcatheter, produce complete occlusion of the aneurysm sac without depending upon thrombosis formation, allow for the formation of a new blood vessel wall over the neck of the aneurysm, and then be absorbed by the body. Disclosed herein is the use of recombinant genetic engineering to combine the environmentally responsive solubility of tropoelastin with the strength of silk fibers to create a bioinspired silk-elastinlike protein polymer (SELP)-based liquid embolic that can be administered via the smallest of microcatheters and occlude CA.

### BRIEF SUMMARY

Disclosed are methods of treating an aneurysm in a subject comprising administering to the subject a therapeutically effective amount of a composition comprising a SELP.

Disclosed are methods of preventing rupture of an aneurysm comprising administering to a subject having an aneurysm a composition comprising a SELP, wherein the SELP is present in the aneurysm and prevents rupture.

Also disclosed are methods of embolizing an aneurysm in a subject comprising administering to the subject a therapeutically effective amount of a composition comprising SELP.

Disclosed are methods of treating AVM in a subject comprising administering to the subject a composition comprising a SELP. In some aspects, the SELP embolizes an abnormal blood vessel in the AVM.

Disclosed are methods of embolizing an AVM in a subject comprising administering to the subject a therapeutically effective amount of a composition comprising a SELP, wherein the SELP embolizes an abnormal blood vessel in the AVM.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a linear amino acid sequence (1 letter amino acid code) of SELP 815K. Amino acids representing the silk like and elastin like blocks are underlined and double underlined respectively, while the lysine substitute is in the rectangle. The tail amino acid sequence is in black.

FIG. 2 shows a temperature response of SELP 815K. The mean storage modulus (bottom two lines) and loss modulus (middle three lines) (n=3) are plotted. Dashed lines represent the 95% confidence interval. The top line indicates the temperature of the system.

FIGS. 3A and 3B show viscosity traces of SELP 815K. A) Shear rate ramp of SELP 815K at 4° C. show shear thinning behavior. Black arrows indicate three shear rates, 0.01 Hz, 120 Hz, and 3000 Hz, exemplifying the shear forces experienced by SELP in the syringe, aneurysm neck, and microcatheter respectively. B) Viscosity measured as a function of temperature from 1-37° C. at 0.1% shear strain and 6.283 rad/s. Plots show the average of 3 samples.

FIGS. 4A and 4B show a microcatheter delivery of SELP embolic. A) The force profiles of SELP embolic, Isovue 370, PVA 300, and Tornado coils injected through a 2.4 Fr microcatheter at 0.5 mL/min shows that SELP requires similar injection force as other clinically used materials. Traces show the average of three distinct runs with the same material. B) SELP 815K injection profile with a 40 s pause to simulate interruptions in the interventionalists administration of the material. After 40 s a fresh syringe with cold saline was used to expel the SELP 815K remaining in the catheter. During the 40 s the SELP 815K started transitioning to a gel, and the force required to discharge the material was somewhat higher.

FIGS. 5A and 5B show an in vitro cytocompatibility of SELP 815K. A) The relative 24 hours viability of L-929 cells when grown in the presence of clinically used commercial materials, controls, and SELP 815K. Triton X (1%) served as the negative control, and no treatment served as the positive control for cell viability. Scale bar represents 50 μm. *** indicates P<0.001 when compared to the no treatment control. †† and ††† indicates P<0.01 and P<0.001 compared to SELP 815K. B) Live/Dead assay of 20 μL SELP 815K disks containing L-929 and HUVEC cells, respectively.

FIG. 6 shows an in vivo angiography of embolization with SELP embolic. The small black circle in each image is a 6.1 mm diameter calibration sphere. In each panel, the black arrow indicates the location of the aneurysm.

FIGS. 7A, 7B, 7C and 7D show a histological examination of the aneurysms using Masson's trichrome special stain. A) Cross-section of the aneurysm of the 8^{th} animal treated with SELP 815K liquid embolic. Administration of 4× aneurysm volume leads to the complete filling of the aneurysm. The arrow indicates the aneurysm generated in the right common carotid artery (RCCA). LCCA: left common carotid artery, DPA: distal parent artery. B) Cross-section of control animal not treated with SELP 815K liquid embolic. The arrow indicates the aneurysm generated in the right common carotid artery (RCCA). LCCA: left common carotid artery, DPA: distal parent artery. C) Cross-section of 4^{th} animal treated with SELP 815K liquid embolic at 3× magnification. Administration of 3.7× aneurysm volume leads to the presence of a neck remnant. The arrow points to the new connective tissue formed across the complete aneurysm neck. D) A 10× magnification of the cross-section shown in panel C. New connective tissue is forming across the complete surface of the SELP embolic and even bridging a gap to form a complete barrier between the aneurysm and the circulating vasculature. Scale bars are as indicated in each image.

FIGS. 8A-8E show a SELP liquid embolic mode of action. A) shows a cerebral aneurysm, the intended treatment target for SELP liquid embolic. B) angiogram still frame of the elastase-induced rabbit animal model pre-procedure. The red box highlights the aneurysm, and the small black circle is a 6.1 mm diameter calibration sphere. C) angiogram still frame of the SELP 815K treated aneurysm. The red box highlights the treated aneurysm, and the small black circle is a 6.1 mm diameter calibration D) shows the insertion of the balloon and microcatheter, the inflation of the balloon, followed by the injection of the SELP liquid embolic. E) shows the physical transformation of the SELP liquid embolic from a solution of protein-polymer strands to a physical gel, followed by the removal of the microcatheter and the balloon.

FIG. 9 shows an example measurement of aneurysm size. The use of angiograms determined aneurysm size and shape.

FIG. 10 shows an example of fluoroscopic imaging of interventional devices and radiopaque SELP embolic.

FIGS. 11A-11D shows viscosity traces of radiopaque embolic formulations. A) Temperature ramp of SELP embolic formulations. B) Shear rate ramp of SELP embolic formulations at 4° C. C) Comparison of SELP formulations at 4° C., 23° C., and 37° C. to represent temperatures the embolic will encounter during its anticipated use. D) Comparison of SELP embolic formulations at shear rates it will encounter during embolization. *, **, *** indicate P<0.001, P<0.05, and P<0.01.

FIGS. 12A-12D show a microcatheter delivery of SELP embolic. A) Injection profile of SELP embolic and clinical materials injected through a 2.4F microcatheter at 0.5 ml/min. shows that SELP is injectable. B) The mean equilibrium injection force±st. dev. of 3 injections through the system. C) SELP injection profile with a pause to connect a syringe with cold saline to push the SELP remaining in the catheter system through the syringe. D) Photograph showing SELP embolic exiting the microcatheter as a liquid.

FIGS. 13A-13D show an example of gelling behavior of SELP. A) Oscilitory time sweep at 37° C. illustrating gelation profiles. B) Oscillatory amplitude sweep at 37° C. of gels occurred for 3 hrs. C) Comparison of gel storage moduli 5 min. and 3 hrs. at 37° C. D) Tilt test visually demonstrating gelation of the materials.

FIGS. 14A, 14B, and 14C show an in vitro biocompatibility of SELP embolic. The relative viability of L-929 of clinical embolics prepared per manufacture's directions after 24 hrs. culture compared to: A) clinically used embolic materials, and B) radiopaque formulations of SELP embolic (n=6). C) Representative images of L-929 and HUVEC cells embedded within SELP embolic while it was still liquid and then allowed to gel. Scale bar represents 50 μm. *** indicates P<0.001 when compared to the no treatment control. †† and ††† indicate P<0.01 and P<0.001 compared to SELP embolic and SELP embolic with contrast. ‡‡‡ indicates P<0.001 for comparisons between SELP with contrast and the contrast alone at equivalent concentrations.

FIG. 15 is a table showing a summary of sterility test findings.

FIG. 16 shows an embolization of a model aneurysm in vitro.

FIG. 17 shows a gross anatomical and histological examination of the aneurysms using Masson's trichrome stain. Scale bars are as indicated in each image. The arrow indicates the aneurysm generated in the right common carotid artery (RCCA). LCCA: left common carotid artery, DPA: distal parent artery.

FIG. 18 shows an example of muscle and brain with and without SELP embolization.

FIG. 19 shows an example of structures of indocyanine green and SELP 815K. A) Illustration of silk-elastinlike protein polymer (SELP) 815K structure. The single letter amino acid code for the protein polymer is listed below the graphic. MW: Molecular Weight. B) Chemical Structure of indocyanine green (ICG).

FIGS. 20A-20C show an effect of ICG on SELP hydrogel properties. A) soluble fractions and B) swelling ratios of SELP 815K hydrogels loaded with ICG. The data represent the mean±st. dev. of n=6 samples. C) SEM images demonstrating lyophilized SELP microstructures with varying ICG concentrations. The scale bars represent 200 μm and 50 μm for the 200× and 1000×, respectively. *: p<0.05, **: p<0.01, ***: p<0.001

FIG. 21 shows an effect of concentration on ICG release from SELP hydrogels. The data represent mean±st. dev. of n=6 samples. ***: p<0.001

FIGS. 22A-22E SELP-ICG viscoelastic properties. A) Viscosity traces of two embolic formulations from 18-37° C., illustrating that temperature increases SELP viscosity and the addition of ICG enhances this effect. B) SELP and SELP-ICG viscosity at 25° C. C) The storage (G′) and loss (G″) moduli of SELP and SELP-ICG over a 3-hr. period demonstrate rapid gelation kinetics and the formation of a robust gel. The dashed lines indicate the 95% confidence interval. D) Storage moduli at 5 min. and 3 hrs. show that ICG incorporation increased the strength of the gel. E) Tilt test of SELP 815K 12 wt/wt % with 0.5 mg/mL of ICG at various times at 37° C. ***p<0.001, The data represent the mean±st. dev. (n=3).

FIGS. 23A and 23B show an example of ICG release and diffusion in agar phantom tissues. A) ICG fluorescence in tissue phantoms shows release and diffusion after simulated embolization. B) BSA enhanced the release of ICG and facilitated diffusion within the tissue phantom and improved fluorescent signal. Partitioning from SELP into the phantom. Data points represent the mean±st. dev. of 6 samples. Comparisons were made between two groups using a 2-tail students T-test of the points at 48 hrs. *p<0.05 and ***p<0.001 between the indicated groups.

FIGS. 24A-24C show visualization of ICG fluorescence. A) ICG is readily visible with a commercially available endoscope, shown as blue overlay, and using IVIS preclinical imaging system, shown in a yellow-hot overlay. However, ICG fluorescence dose not directly correlate with concentration. B) Image analysis demonstrates that there is visually apparent self-quenching that occurs at concentrations higher than 0.012 mg/mL ICG. C) Signal from IVIS and the endoscope are directly proportional.

FIGS. 25A and 25B show SELP-ICG embolization and visualization in a microfluidic model tumor. A) Graphical illustration of embolization test setup with images of microfluidic models before and after embolization. After embolization, there was no perfusion to the embolized chip. Below the illustration is a magnified version of one of the collateral flow chips filled with methylene blue to show channel structure. B) IVIS images of the tumor microfluidic chip (top) and a collateral flow chip (bottom) show fluorescence within the SELP-ICG embolized tumor chip but not in the collateral chips.

FIG. 26 shows computational modeling of shear-force of simulated blood flowing through microfluidic tumor models. Color gradient represents the shear force experienced by the fluid for: A) 1st Generation, D) 2nd Generation, and C) 3rd Generation designs. Images and models were generated using Comsol Multiphysics 5.4. The designs were developed to reduce turbulent flow and reduce dead space within the structures.

FIGS. 27A and 27B show pressure vs. flow rate through 3 microfluidic tumor models plumbed in parallel. A) Pressure profiles with a flow rate ramp using PBS for the 3rd generation design of microfluidic tumor model. B) Flow rate vs. pressure showed the anticipated linear relationship. The dashed line indicates the regression line of the flow profile. Each point represents the average of 10 sec. of data taken from the equilibrium pressure of the system at each flow rate.

FIGS. 28A and 28B Cytotoxicity of ICG and SELP-ICG. A) L929 fibroblast and B) HUVEC viability curves in response to increasing ICG concentration of ICG alone or SELP-ICG. The data represent the mean±st. dev. of 6 samples. The solid lines represent the curve derived from fitting the data to a variable slope Hill equation.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a SELP” includes a plurality of such SELPs, reference to “the SELP” is a reference to one or more SELPs and equivalents thereof known to those skilled in the art, and so forth.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

A “hydrogel” as used herein refers to a semisolid composition constituting a substantial amount of water. A hydrogel can be formed from a network of polymer chains in which polymers or mixtures thereof are dissolved or dispersed. Hydrogels are composed of three dimensional polymer networks that will swell without dissolving when placed in water or other biological fluids. A hydrogel is significantly more viscous than water or other similar liquids. Hence, for purposes herein, a hydrogel is generally a non-liquid form.

By “treat” is meant to administer a composition or SELP of the invention to a subject, such as a human or other mammal (for example, an animal model), that has a CA, in order to prevent or delay a worsening of the effects of the CA, or to partially or fully reverse the effects of the CA.

The term “prevent” as used herein is defined as eliminating or reducing the likelihood of the occurrence of one or more symptoms of a disease or disorder (e.g., CA) when compared to the same symptom in the absence of the compound.

By an “effective amount” of a composition or SELP as provided herein is meant a sufficient amount of the composition or SELP to provide the desired effect. The exact amount required will vary from subject to subject, depending on the species, age, and general condition of the subject, the severity of disease (e.g., CA) that is being treated, the particular composition used, its mode of administration, and the like. Thus, it is not possible to specify an exact “effective amount.” However, an appropriate “effective amount” may be determined by one of ordinary skill in the art using only routine experimentation. In some aspects, effective amount depends upon the rate of injection and how long the polymer is allowed to rest prior to administration. Altering, the timing of the administration can be used to control the depth of penetration of the SELP embolic. The term “therapeutically effective amount” means an amount of a therapeutic, prophylactic, and/or diagnostic agent that is sufficient, when administered to a subject suffering from or susceptible to a disease, disorder, and/or condition (e.g. CA), to treat, alleviate, ameliorate, relieve, alleviate symptoms of, prevent, delay onset of, inhibit progression of, reduce severity of, and/or reduce incidence of the CA.

As used herein, the terms “administering” and “administration” refer to any method of providing a disclosed SELP, composition, or a pharmaceutical composition to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, interstitial administration, and subcutaneous administration. Administration can be continuous or intermittent. Administration can be through a syringe, catheter, microcatheter, nose, needle, or other geometry. Surgery coupled with local injection into a nidus or sac of a vascular abnormality could be used to introduce the embolic into the luminal space. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed SELP so as to treat a subject or cause embolization. In an aspect, the skilled person can also alter or modify an aspect of an administering step so as to improve efficacy of a disclosed SELP, composition, or a pharmaceutical composition.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

“Embolic” or embolics” as used herein refers to a composition composition capable of causing or inducing an embolism. For example, an embolic can be a coil, gelfoam, particle, or liquid sclerosants. Additional embolics include those described in Golzarian et al. “An Overview of Embolics”, Endovascular Today, April (2009) 37-41, which is hereby incorporated by reference in its entirety for teaching embolics. In some aspects, the embolic is a SELP embolic. In some aspects, the embolic is Butyl cyanoacrylate (NBCA), ethiodol, ethanol, ethanolamine oleate, sotradecol, polyvinyl alcohol (PVA), Embolization microspheres, or a tissue adhesive.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Silk-Elastinlike Polymers (SELPs)

The compositions described herein include a silk-elastinlike protein (SELP). SELPs are a class of genetically engineered protein polymers composed of repeating “blocks” of amino acids, referred to as “silk blocks” (Gly-Ala-Gly-Ala-Gly-Ser) and “elastin blocks” (Gly-Val-Gly-Val-Pro). By varying the number of silk and elastin blocks, the rheological properties of the composition can be modified to fit specific applications. For example, the silk-to-elastin ratio and the length of the silk and elastin block domains as well as the SELP concentration can be modified to optimize gelling upon administration of the composition to a subject. Any of the disclosed SELPs can be used in the methods disclosed herein.

Examples of SELPs useful herein include, but are not limited to,

[(VPGVG)8(GAGAGS)2]18;
[(GVGVP)4(GAGAGS)9]13;
[(VPGVG)8(GAGAGS)4]12;
[(VPGVG)8(GAGAGS)6]12;
[(VPGVG)8(GAGAGS)8]11;
[(VPGVG)12(GAGAGS)8]8;
[(VPGVG)16(GAGAGS)8]7;
[(VPGVG)32(GAGAGS)8]5;
[(GAGAGS)12GAAVTGRGDSPASAAGY(GAGAGS)5(GVGVGP)8]6;
[(GAGAGS)2(GVGVP)4GKGVP(GVGVP)3]6;
[(GAGAGS)2(GVGVP)4GKGVP(GVGVP)3]12;
[(GAGAGS)2(GVGVP)4GKGVP(GVGVP)3]18;
[(GAGAGS)2(GVGVP)4GKGVP(GVGVP)3]17(GAGAGS)2;
[(GAGAGS)2(GVGVP)4GKGVP(GVGVP)3(GAGAGS)2]13;
[GAGAGS(GVGVP)4GKGVP(GVGVP)3(GAGAGS)2]12;
[(GVGVP)4GKGVP(GVGVP)11(GAGAGS)4]5(GVGVP)4GKGVP(GVGVP)11(GAGAGS)2;
[(GVGVP)4GKGVP(GVGVP)11(GAGAGS)4]7(GVGVP)4GKGVP(GVGVP)11(GAGAGS)2;
[(GVGVP)4GKGVP(GVGVP)11(GAGAGS)4]9(GVGVP)4GKGVP(GVGVP)11(GAGAGS)2;
[GAGS(GAGAGS)2(GVGVP)4GKGVP(GVGVP)11(GAGAGS)5GA]6;
[(GAGAGS)2GVGVPLGPLGP(GVGVP)3GKGVP(GVGVP)3]15(GAGAGS)2;
[(GAGAGS)2GVGVPGFFVRARR(GVGVP)3GKGVP(GVGVP)3]15(GAGAGS)2.

In some aspects, the SELP can be

MDPVVLQRRDWENPGVTQLNRLAAHPPFASDPM[GAGS
(GAGAGS)₂(GVGVP)₄GKGVP(GVGVP)₁₁(GAGAGS)₅GA]₆
GAMDPGRYQDLRSHHHHHH(SELP-815K)
or
MDPVVLQQRDWENPGVTQLVRLAAHPPFASDPMGAGSGAGAGS
[(GVGVP)₄GKGVP(GVGVP)₃(GAGAGS)₄]₁₂(GVGVP)₄GKGVP
(GVGVP)₂(GAGAGS)₂GAMDPGRYQDLRSHHHHHH(SELP-47K)

The underlined sequences are tail sequences or cloning scars. The tail sequences, or cloning scars, can aid in expression, solubilization, stabilization, and/or purification.

In some aspects, the SELP can be [GAGS(GAGAGS)ₙ₁(GVGVP)ₙ₂GXGVP(GVGVP)ₙ₃(GAGAGS)ₙ₄GA]ₙ₅GA, wherein X can be any amino acid, and wherein n1, n2, n3, n4, and n5 can each be any number ranging from 1-100.

In some aspects, X can be any hydrophilic amino acid, such as, but not limited to glutamine, asparagine, histidine, serine, threonine, tyrosine, and cysteine. In some aspects, X can be any cationic amino acid, such as, but not limited to, lysine, arginine, histidine. In some aspects, X can be any amino acid eligible for bioconjugation. For example, an amino acid eligible for bioconjugation can be, but is not limited to, lysine, cystine, tyrosine, glutamatic acid, aspartic acid, tryptophan, arginine, and histidine.

In some aspects, n1 can be any number ranging from 2-10, n2 can be any number ranging from 1-50, n3 can be any number ranging from 1-50, n4 can be any number ranging from 2-10, and n5 can be any number ranging from 1-14. In some aspects, n2+n3+1 must be greater than 7 but less than 100. In some aspects, n1+n4 must be greater than 2 but less than 20. Thus, for example, the disclosed SELPs comprise at least 7 elastin blocks and at least 2 silk blocks. In some aspects, the SELP comprises more elastin blocks than silk blocks.

In some aspects, the SELP comprises the sequence of

[GAGS(GAGAGS)₂(GVGVP)₄GKGVP(GVGVP)₁₁(GAGAGS)₅GA]₆GA.

In another aspect, the silk-elastinlike polymer can be a variant of a SELP. A “variant” with reference to a silk-like unit or elastin-like unit refers to a silk-like unit or elastin-like unit that has an amino acid sequence that is altered by one or more amino acids. Typically, a unit sequence is altered by 1, 2, or 3 amino acids. The variant can have an amino acid replacement(s), deletion(s), or insertion(s). For example, the variant can have “conservative” changes, wherein a substituted amino acid has similar structural or chemical properties (e.g., replacement of valine with isoleucine). In some cases, a variant can have “nonconservative” changes (e.g. replacement of a glycine with a tryptophan). Similar minor variations can also include amino acid deletions or insertions, or both. In addition to the teachings herein, guidance in determining which amino acid residues can be substituted, inserted, or deleted without abolishing bioactivity can be found using computer programs well known in the art such as, for example, DNASTAR software.

In one aspect, the SELP is sheared. In one aspect, a solution of the SELP is introduced into a homogenizer through a needle valve at a pressure of from 1,500 psi to 17,000 psi. Exemplary methods for producing sheared SELPs are provided in Price et al, “Effect of shear on physicochemical properties of matrix metalloproteinase responsive silk-elastinlike hydrogels,” J. Control. Release, 2014, 195:92-98. Not wishing to be bound by theory, the shearing of the SELP solution breaks intramolecular hydrogen bonds between the silk-like motifs. Shearing linearizes the protein, which causes reduction in solution viscosity and increases the opportunity for the formation of intermolecular interactions between the silk-like domains of distinct SELP polymers. Shearing can ultimately increase the peak modulus and gelation rate of the SELP. Increased intermolecular bonding enables the formation of a stiffer and more homogeneous network.

### C. Compositions

Disclosed are compositions comprising one or more of the disclosed SELPs.

1. Pharmaceutical Compositions

In some aspects, the disclosed compositions can be pharmaceutical compositions. For example, in some aspects, disclosed are pharmaceutical compositions comprising a composition comprising a SELP and a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, or conjugate of the invention is not compromised. Pharmaceutical compositions may also include one or more active ingredients (in addition to the composition of the invention) such as antimicrobial agents, anti-inflammatory agents, anesthetics, and the like.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the fusion proteins. Thus, compositions can be prepared for parenteral administration that includes fusion proteins dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The pharmaceutical compositions described above can be formulated to include a therapeutically effective amount of a composition disclosed herein. In some aspects, therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to one or more autoimmune diseases or where the patient has a clinically determined predisposition or increased susceptibility (in some cases, a greatly increased susceptibility) to cancer.

The pharmaceutical compositions described herein can be administered to the subject (e.g., a human subject or human patient) in an amount sufficient to delay, reduce, or preferably prevent the onset of clinical disease. Accordingly, in some aspects, the subject is a human subject. In therapeutic applications, compositions are administered to a subject (e.g., a human subject) already with or diagnosed with an autoimmune disease in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effective amount includes amounts that provide a treatment in which the onset or progression of the cancer is delayed, hindered, or prevented, or the autoimmune disease or a symptom of the autoimmune disease is ameliorated. One or more of the symptoms can be less severe. Recovery can be accelerated in an individual who has been treated.

The total effective amount of the conjugates in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

The pharmaceutical composition may be administered in a number of ways depending on whether local or systemic treatment is desired, and on the area to be treated.

### D. Methods

1. Aneurysms

Disclosed are methods of treating aneurysms. Aneurysms can comprise saccular, fusiform, dissected, and false aneurysms. Saccular aneurysms can occur in several places, including but not limited to, the brain, neck, leg, and kidney. Saccular, dissected and false aneurysms can be treated in a similar manner. Each of these aneurysms comprise a void on one side of an artery that can be filled by SELP using a balloon occlusion of the aneurysm neck prior to filling the void with the SELP. Fusiform aneurysms comprise a void on both sides of an artery wherein treatment with stents are used. SELPs can be used in fusiform aneurysms to help fill voids left behind after placement of a stent.

Disclosed are methods of treating an aneurysm in a subject comprising administering to the subject a therapeutically effective amount of a composition comprising a SELP.

Disclosed are methods of preventing rupture of an aneurysm comprising administering to a subject having an aneurysm a composition comprising a SELP, wherein the SELP is present in the aneurysm and prevents rupture.

Also disclosed are methods of embolizing an aneurysm in a subject comprising administering to the subject a therapeutically effective amount of a composition comprising SELP.

Any of the disclosed SELPs or compositions comprising a SELP can be used in the disclosed methods. In some aspects, the SELP can be [GAGS(GAGAGS)ₙ₁(GVGVP)ₙ₂GXGVP(GVGVP)ₙ₃(GAGAGS)ₙ₄GA]ₙ₅GA, wherein X can be any amino acid, wherein n1 can be any number ranging from 2-10, n2 can be any number ranging from 1-50, n3 can be any number ranging from 1-50, n4 can be any number ranging from 2-10, and n5 can be any number ranging from 1-14. In some aspects, the SELP comprises the sequence of [GAGS(GAGAGS)₂(GVGVP)₄GKGVP(GVGVP)₁₁(GAGAGS)₅GA]₆GA. In some aspects, the composition can further comprise a pharmaceutically acceptable carrier. Thus, the disclosed methods can use a pharmaceutical composition comprising any of the disclosed compositions or SELPs.

In some aspects, the aneurysm can be a saccular aneurysm. In some aspects, the saccular aneurysm can be a cerebral aneurysm (CA).

In some aspects, the subject has been diagnosed with an aneurysm.

In some aspects, the SELP embolizes the aneurysm.

In some aspects, the SELP transitions from a liquid to a hydrogel at temperatures above 23° C. For example, the transition from room temperature (23° C.) to body temperature (37° C.) results in a shift from a liquid state to a solid gel.

In some aspects, a therapeutically effective amount is at least 1× the aneurysm volume. In some aspects, the therapeutically effective amount is at least 2× the aneurysm volume. In some aspects, the therapeutically effective amount is at least 3× the aneurysm volume. In some aspects, the therapeutically effective amount is at least 4× the aneurysm volume.

In some aspects, the composition is administered using a catheter. A catheter can be used in combination with balloon occlusion. In some aspects, aneurysms can comprise an aneurysm neck and an aneurysmal sac. In some aspects, balloon occlusion can be used to block the aneurysm neck so that the catheter can direct the SELP into the aneurysmal sac wherein the SELP can fill the void within the sac. In some aspects, fill the void within the sac can mean completely fill the void or partially fill the void. Thus, in some aspects, the composition comprising the SELP is administered into or enters the aneurysmal sac. The SELP, once it forms the hydrogel, can comprise at least a quarter, a half, or three-quarters of the aneurysmal sac. In some aspects, the entire aneurysmal sac is filled with the SELP hydrogel.

In some aspects, no distal embolisms are present. Several embolics on the market have the adverse effect that if the embolic migrates away from the aneurysm it can cause an embolism elsewhere in the body. In some aspects, if the disclosed SELPs dilute causing a few of the SELP polymers to migrate away from the aneurysm, they will not cause a distal embolism elsewhere in the body. This provides an added safety mechanism for the disclosed methods.

In some aspects, the SELP remains in the aneurysm for one month. In some aspects, the SELP remains in the aneurysm for days, weeks, months or years. In some aspects, the SELP remains in the aneurysm for 1, 2, 3, 4, 5, 6, or 7 days. In some aspects, the SELP remains in the aneurysm for 1, 2, 3, or 4 weeks. In some aspects, the SELP remains in the aneurysm for 1, 2, 3, 4, 5, 6, 7, 8, 9, 10, 11, or 12 months. In some aspects, the SELP remains in the aneurysm for 1, 2, 3, 4, 5, 6, 7, 8, 9, or 10 years.

In some aspects, the disclosed composition further comprises a contrast agent. For example, a contrast agent can be, but is not limited to, micronized tantalum or an iodine based contrast. Examples of iodine based contrasts can be, but are not limited to, Iodixanol, iopamidol Ithalamate, iohexol, ioversol, iopromide, diatriazoate.

In some aspects, the composition further comprises a visualization agent. For example, a visualization agent can be, but is not limited to, a dye or a fluorophore.

In some aspects, the composition further comprises a therapeutic agent. For example, a therapeutic agent can be, but is not limited to, a growth factor, extracellular matrix (ECM) protein, or pro-clotting factor. In some aspects, a therapeutic agent helps promote healing and closure of the aneurysm sac.

2. Arteriovenous Malformations (AVM)

Arteriovenous malformations (AVM) occurs when arteries and veins are not formed correctly in an area of the body. AVM is an abnormal tangle of blood vessels connecting arteries and veins. Disclosed are methods of treating AVM. In some aspects, SELPs can be used to embolize the blood vessel that feeds the AVM.

Disclosed are methods of treating AVM in a subject comprising administering to the subject a composition comprising a SELP. In some aspects, the SELP embolizes an abnormal blood vessel in the AVM.

Disclosed are methods of embolizing an AVM in a subject comprising administering to the subject a therapeutically effective amount of a composition comprising a SELP, wherein the SELP embolizes an abnormal blood vessel in the AVM.

In some aspects, the subject has been diagnosed with AVM.

Any of the disclosed SELPs or compositions comprising a SELP can be used in the disclosed methods. In some aspects, the SELP can be [GAGS(GAGAGS)n1(GVGVP)n2GXGVP(GVGVP)n3(GAGAGS)n4GA]n5GA, wherein X can be any amino acid, wherein n1 can be any number ranging from 2-10, n2 can be any number ranging from 1-50, n3 can be any number ranging from 1-50, n4 can be any number ranging from 2-10, and n5 can be any number ranging from 1-14. In some aspects, the SELP comprises the sequence of [GAGS(GAGAGS)2(GVGVP)4GKGVP(GVGVP)11(GAGAGS)5GA]6GA. In some aspects, the composition can further comprise a pharmaceutically acceptable carrier. Thus, the disclosed methods can use a pharmaceutical composition comprising any of the disclosed compositions or SELPs.

In some aspects, the SELP transitions from a liquid to a hydrogel at temperatures above 23° C. For example, the transition from room temperature (23° C.) to body temperature (37° C.) results in a shift from a liquid state to a solid gel.

In some aspects, a therapeutically effective amount is at least 1× the aneurysm volume. In some aspects, the therapeutically effective amount is at least 2× the aneurysm volume. In some aspects, the therapeutically effective amount is at least 3× the aneurysm volume. In some aspects, the therapeutically effective amount is at least 4× the aneurysm volume.

In some aspects, the composition is administered using a catheter. A catheter can be used in combination with balloon occlusion or in combination with other tools such as stents, or other flow restricting devices.

In some aspects, no distal embolisms are present. Several embolics on the market have the adverse effect that if the embolic migrates away from point of interest (e.g. the AVM) it can cause an embolism elsewhere in the body. In some aspects, if the disclosed SELPs dilute causing a few of the SELP polymers to migrate away from the AVM, they will not cause a distal embolism elsewhere in the body. This provides an added safety mechanism for the disclosed methods.

In some aspects, the SELP remains in the AVM for one month. In some aspects, the SELP remains in the AVM for days, weeks, months or years. In some aspects, the SELP remains in the AVM for 1, 2, 3, 4, 5, 6, or 7 days. In some aspects, the SELP remains in the AVM for 1, 2, 3, or 4 weeks. In some aspects, the SELP remains in the AVM for 1, 2, 3, 4, 5, 6, 7, 8, 9, 10, 11, or 12 months. In some aspects, the SELP remains in the AVM for 1, 2, 3, 4, 5, 6, 7, 8, 9, or 10 years.

In some aspects, the disclosed composition further comprises a contrast agent. For example, a contrast agent can be, but is not limited to, micronized tantalum or an iodine based contrast. Examples of iodine based contrasts can be, but are not limited to, Iodixanol, iopamidol Ithalamate, iohexol, ioversol, iopromide, diatriazoate.

In some aspects, the composition further comprises a visualization agent. For example, a visualization agent can be, but is not limited to, a dye or a fluorophore.

In some aspects, the composition further comprises a therapeutic agent. For example, a therapeutic agent can be, but is not limited to, a growth factor, extracellular matrix (ECM) protein, or pro-clotting factor.

3. A Dual-Functional Embolization-Visualization System for Fluorescence Image-Guided Surgery

The concept that an interventionist can use embolics to enable florescent-guided surgery is an actively growing field. Fluorescent labels right now are allowed to diffuse throughout the body and then accumulate through various means. However, the field is plagued by low accumulation and poor contrast between tissue types. Having an interventionalist who is actively using imaging to guide and perform tissues. Many of the imaging techniques used by the interventionalists (particular x-ray-based technology, MRI, PET, etc.) are not able to be used during other types of surgery (open, endoscopic, robotic, etc.). If they can use embolics to place markers that the surgeon can see during their procedure the interventionalist can very effectively convey information on location of arteries and or margins of the malignancy.

Thus, disclosed are methods comprising administering an embolic to a subject in need thereof, wherein the embolic is conjugated to a visualization agent, wherein the embolic causes embolization and the visualization agent allows a surgical site to be identified. In some aspects the surgical site is a tumor.

In some aspects, the embolic can be one or more of the SELPs disclosed throughout.

In some aspects, the surgery is a surgery to resect a tumor. For example, the SELP can reduce intraoperative bleeding by causing an embolization at the tumor while at the same time deliver a visualization agent that demarcates the tumor margins. This process allows the tumor to be better visualized during surgery.

In some aspects, the subject in need thereof is a subject that needs surgery. For example, a cancer patient can be a subject in need thereof because they may need a tumor surgically removed.

Disclosed herein are methods comprising administering an embolic to a subject in need thereof, wherein the embolic is conjugated to a visualization agent. In some aspects, the embolic is a SELP embolic. In some aspects, the embolic is a coil, gelfoam, particle, or liquid sclerosants.

In some aspects, the embolic is Butyl cyanoacrylate (NBCA), ethiodol, ethanol, ethanolamine oleate, sotradecol, polyvinyl alcohol (PVA), Embolization microspheres, or a tissue adhesive.

A method comprising administering an embolic to a subject in need thereof, wherein the embolic is conjugated to a visualization agent, wherein the embolic causes embolization and the visualization agent allows a surgical site to be identified.

A method of identifying or labeling a surgical site in a subject comprising administering an embolic to the subject, wherein the embolic is conjugated to a visualization agent.

### EXAMPLES

### A. Example 1

1. Introduction

Cerebral aneurysms (CA) rupture spontaneously and are the primary cause of severe hemorrhagic stroke. CA is a bulge in a weakened blood vessel wall that is present in 3.2% of the general population and is among the most common types of vascular malformations. Aneurysm rupture is fatal in 50% of cases and causes severe disability in over 50% of survivors. CAs are especially challenging to treat in part due to the risks associated with damaging nearby healthy tissues during the intervention.

Current therapies prevent rupture by diverting flow away from the aneurysm either by filling the aneurysmal sac with an embolic material or diverting flow using a stent-like device. However, these treatments fail due to recanalization in 20-57% of cases. An ideal treatment for CA would be minimally invasive, reinforce weakened vasculature, reduce shear forces on aneurysmal wall, and facilitate healing of the weakened vasculature. While embolization and flow diversion are the standards of care to prevent intracranial hemorrhage in high-risk patients, metal embolization coils and flow diversion devices require catheters that can accommodate the diameter of the device during delivery and anticoagulation therapy, cause artifacts on follow-up imaging via magnetic resonance imaging (MRI) or computed tomography imaging (CT), induce thrombus in undesirable locations, and can undergo recanalization. Liquid embolics have the potential to fill an aneurysm completely, but current liquid embolics are challenging to use due to their high viscosity, limited selection of compatible catheters, and dependence on potentially toxic organic solvents. Next-generation liquid embolics should have the advantages of not using potentially toxic organic solvents, have low viscosity to allow flow through small-diameter microcatheters, and have the capacity to carry various classes of therapeutics while providing durable embolization of the target lumen. Such an embolic would reduce mechanical strain by blocking flow to the aneurysmal sac and prevent the aneurysm from growing by reinforcing weakened vasculature.

One way to develop new liquid embolics is to use temperature-responsive protein-based polymers. Protein-based polymers have well-controlled structures derived from genetic instructions that define monomer sequence and molecular weight, allowing for the precise tailoring of structure to meet functional requirements. Silk-elastinlike protein polymers (SELPs) are one class of protein-based polymers that combine the solubility of mammalian elastin and the strength of silk to create macromolecules with tunable solubility and mechanical properties. Rational design of the ratio and sequence of silk and elastin motifs, polymer length, and concentration in solution dictate properties such as gelation rate, mechanical rigidity, and network density. Depending on sequence and length, many SELPs, when dissolved in phosphate-buffered saline (PBS), remain as injectable solutions at room temperature, pass through catheters without occluding, and rapidly transition to a solid hydrogel after injection. SELPs have demonstrated in vivo stability for greater than 12 weeks and have shown no evidence of toxicity or excessive inflammation.

SELPs can be liquid embolics. SELP compositions demonstrated acceptable rheological properties and clear embolic capability under flow conditions in vitro. In a rabbit model, selective occlusion of lobar hepatic arterial branches was shown. Described herein is the utility of SELPs as liquid embolics for occlusion of CA.

2. Materials and Methods

i. Production of SELP Embolic

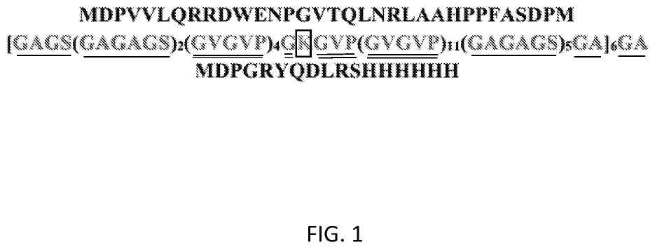

SELP 815K, structure shown in FIG. 1, which contains 8 silk-like motifs, 15 elastin-like motifs, and 1 lysine-substituted elastinlike motif per monomer repeat, was produced by expression in E. coli from a recombinant plasmid. Production was performed by previously reported procedures but scaled up to accommodate 10 L and 100 L batches. The SELP 815K was purified from the crude biomass and sheared as a 12% (w/w) solution in accordance with previously described methods with the addition of 316 stainless steel cooling loop submerged in 0-4° C. water bath and UV sterilization via a PHRED™ reactor (Aura Industries Inc., San Diego, Calif.) after the high-pressure homogenizer.

ii. Rheology

SELP 815K temperature response was characterized using a TA 550 stress-controlled rheometer (TA Instruments, New Castle, Del.) with a stainless steel 4°, 20 mm diameter cone and plate geometry. An oscillatory sweep was performed at 6.283 rad/s and 0.1% strain. The temperature was held at 23° C. for 30 min before it was increased (10° C./min) to 37° C. and held for 1 hour. Subsequent rheology to analyze viscosity and gelation kinetics of SELP 815K was conducted on a Malvern Kinexus Ultra+ Rheometer (Malvern Panalytical Ltd, Egham, Surrey, United Kingdom) with a 2°, 20 mm stainless steel cone and plate geometry. An active solvent trap was placed around the periphery of the plate to reduce water loss due to evaporation during testing.

Viscosity was measured from 1 to 37° C. (5° C./min) using an oscillatory procedure at an angular frequency of 6.283 rad/s, immediately followed by a 3-hour oscillatory sweep at 37° C. using 0.1% strain and an angular frequency of 6.283 rad/s to monitor gelation kinetics as well as G′ and G″. All tests were performed at least in triplicate.

iii. In Vitro Injection Testing

Injection testing was used to assess the injectability of SELP 815K and to compare it to clinically used devices. Catheter injections were made using Harvard Apparatus 22 V008 syringe pump (Harvard Apparatus, Holliston, Mass.) outfitted with a low profile USB output load cell (Omega Engineering, Karvina, Czech Republic). The Omega Digital Transducer software version 2.3.0. recorded the signal from the load cell. PVA-300 Foam Embolization Particles (Cook Medical LLC., Bloomington, Ind.), a Tornado® Embolization Coil (Cook Medical LLC., Bloomington, Ind.), and Isovue 370 (Bracco Diagnostics Inc., Monroe Township, N.J.) were used as references for the injection force of approved devices. Injections were performed using 1 mL BD syringes at a rate of 0.5 mL/min through a 2.4 Fr 150 cm long Merit Maestro Microcatheter submerged in a 37° C. water bath. A holder was used to ensure that each catheter was in a consistent position between tests. Clinical embolics were prepared according to manufacturer instructions and administered through the catheter, as described above. The catheter was flushed with cold saline prior to SELP injection. After the complete syringe of SELP 815K liquid embolic was injected, a syringe filled with cold saline was used to push the remaining SELP from the catheter.

iv. Cytotoxicity

L-929 murine fibroblast cell line (American Type Culture Collection, Manassas, Va.), selected for their recommendation by the U.S. Food and Drug Administration for cytotoxicity testing were cultured and seeded into 96-well plates (Thermo Fisher Scientific, Waltham, Mass.) as previously described. 1% Pen-Strep (Thermo Fisher Scientific, Waltham, Mass.) was added to the media. Cell viability was measured after 24 hours using a Cell Counting Kit-8 (CCK-8) (Dojindo, Kumamoto, Japan) per manufacturer's directions. Dulbecco's Phosphate-Buffered Saline (DPBS) and 1% Triton-X (Sigma-Aldrich, St. Louis, Mo.) were used as negative and positive controls, respectively. Clinically available embolic materials and contrast agents including Onyx® 18 (ev3 Inc., Plymouth, Minn.), Quadrasphere® (Merit Medical, South Jordan, Utah), PVA-300 microspheres (Cook Medical, Bloomington, Ind.), Isovue® 300 (Bracco, Milan, Italy) and Conray® 60% (Liebel-Flarsheim Company LLC, Raleigh, N.C.) were used to provide additional context.

To test cell viability, when encapsulated in SELP 815K, human umbilical vein endothelial cells (HUVEC) and L-929 cells were separately mixed into freshly thawed SELP 815K to create 10⁶ cells/mL suspensions. The SELP-cell mixtures were then loaded into tuberculin syringes and incubated at 37° C. for 30 min. The ends of the syringes were removed, the solid SELP-cell cylinders pushed out, and the resulting cylinders sectioned into 20 μL disks. The disks were placed, one disk per well, in 6 well cell culture plates (Thermo Fisher Scientific, Waltham, Mass.). Each well had 3 mL of media added, and the media was replaced every 48 hr. The cell culture plates were kept on rocker tables within a 5% CO₂ incubator at 37° C. Positive control gels were incubated in media with 0.1% Triton-X for 30 min at 37° C. to kill the cells. Live/dead cell viability assay (Thermo Fisher Scientific, Waltham, Mass.) was used to stain cells within the gels prior to imaging on an FV1000 Olympus Confocal Microscope using the manufacturer's recommended settings.

v. In Vivo Testing of SELP Embolic in a Rabbit Cerebral Aneurysm Model

A pilot study was conducted at North American Science Associates, Inc. (NAMSA) to assess the embolic potential of SELP 815K. Animals received daily health and behavioral assessments by trained personnel throughout the conduct of the study.

Aneurysms were generated in 15 New Zealand White rabbits in the right common carotid artery (RCCA). Aneurysm generation involved surgically isolating the RCCA, advancing a balloon catheter to the origin of the RCCA through a vascular sheath, inflating the balloon catheter to block flow into the RCCA, injecting Porcine Elastase (50 U/mL) into the RCCA, and incubating for 20 minutes to induce aneurysm formation. The balloon catheter was then deflated and removed, the vessel was rinsed with saline, the sheath and microcatheter were removed, and the distal RCCA was ligated. The embolization of the aneurysm models was performed 30-31 days after aneurysm creation to allow for the model aneurysm to mature and the animals to recover.

The right femoral artery was surgically accessed, a 6 Fr radial sheath was inserted into the femoral artery, and a bolus injection of heparin administered. A Cordis MPA 6F×100 cm guide catheter was directed under fluoroscopy to near the origin of the RCCA. A Transform® Compliant Occlusion Balloon Catheter (4 mm×10 mm×150 cm) (Stryker, Kalamazoo, Mich.) was placed near the aneurysm neck. An Excelsior® SL-10 microcatheter (150 cm long, 1.7 Fr, 0.60 mm) (Stryker, Kalamazoo, Mich.) was placed within the aneurysm. Angiography was performed to measure the aneurysm dimensions, and an ellipsoid model was used to calculate the volume of the aneurysm.

Prior to injection, the syringe with SELP 815 K was thawed in sterile saline. The injection volume of SELP was initially 1×, the estimated aneurysm volume plus the catheter hold up volume, but was gradually increased to 4×, four times the aneurysm volume plus the catheter hold up volume until the follow-up angiography showed >90% filling of the aneurysmal sac. Time of balloon occlusion was reduced from 30 min to 10 min after thorough occlusion of the aneurysm was established at the more extended time point. Slight negative pressure was applied to the microcatheter before it was retrieved past the occluding balloon. The balloon catheter was left in place to allow the SELP to solidify in the aneurysm. After the balloon catheter was removed, angiography was performed again, and the volume of occlusion visually assessed. Thirty days after initial embolization, the left femoral artery was surgically accessed, a guide catheter was used to deliver contrast, and angiography was performed to evaluate the occlusion of the aneurysm.

vi. Gross Evaluation of Embolization Via Necropsy

Macroscopic examination of the animals was performed by a veterinarian during necropsy. The aneurysm and surrounding vasculature were isolated, inspected, and photographed. Other tissues were examined and evaluated for lesions or any other signs of adverse effects. The right forelimb, brain, and the aneurysm with surrounding vasculature were collected and stored in 10% neutral buffered formalin for a minimum of 24 hours to achieve fixation.

vii. Histology

The aneurysm and surrounding vasculature were processed into a single block to provide anatomical context to the histology. At least two sections of the supraspinatus and subscapularis muscles from the right forelimb and two sections from the brain were obtained from each animal and processed for the evaluation of off-target effects, as these tissues are down-stream from the site of embolization. Each set of tissue sections was embedded in paraffin, sectioned into 5 μm slices, and stained with hematoxylin and eosin (H&E) by the research histology core at the University of Utah Huntsman Cancer Institute core facility. The aneurysms and associated vasculature were additionally stained using Masson's trichrome special stain.

viii. Statistics

Data were recorded, organized, and processed using Excel® (Microsoft, Redmond, Wash.). GraphPad Prism 5.0 was used to perform statistical analysis. All numerical data in this manuscript were assumed to be parametric in nature. One-way analysis of variance (ANOVA) with a posthoc Bonferroni multiple comparison test of data sets with 3 or more groups was used. A p-value of less than 0.05 was used as the threshold to ascribe statistical significance to a result.

3. Results

i. SELP 815K Gel Formation

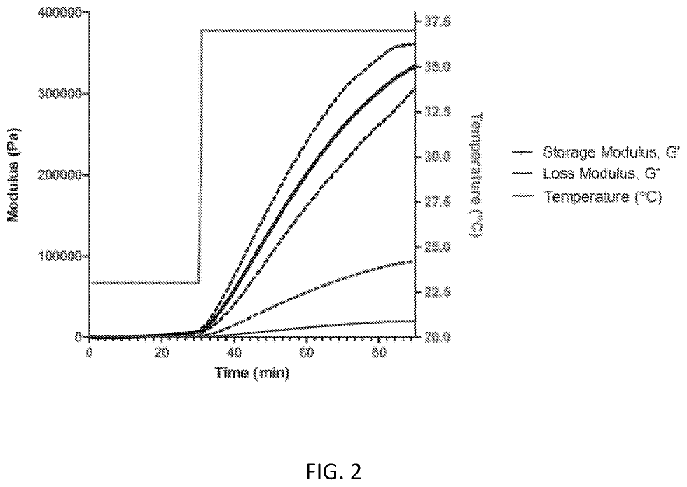

SELP 815K self-assembly responds dynamically to temperature. Elevation from room temperature (23° C.) to 37° C. initiated a rapid shift from a liquid state to a solid gel (FIG. 2). The growing separation between the storage and loss modulus is indicative of the transition to an increasingly stiff material. Transitioning from 23° C. to 37° C. changed the storage modulus over 5.5 logs in magnitude

ii. Shear Thinning Behavior of SELP 815K

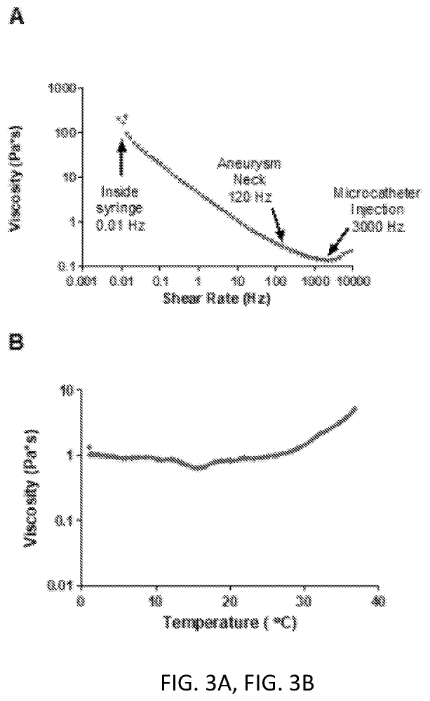

SELP 815K demonstrates shear thinning behavior (FIG. 3A). As the shear rate increases and exceeds the intermolecular interactions between SELP chains, the viscosity of the system decreases. Newtonian fluids will experience a 3000 Hz shear rate during a 0.5 mL/min injection through a 1.8 Fr (0.60 mm) diameter catheter and ˜120 Hz shear rate at the aneurysm neck. SELP 815K has a viscosity of 0.30 and 0.14 Pas at 120 and 3000 Hz, respectively. Viscosity measured at 0.1% shear strain shows minor variations over 1-37° C. (FIG. 3B). Together, the shear thinning behavior and low viscosity variation over the temperature range show that SELP is easily injectable by hand under expected in vivo conditions.

iii. Injectability of SELP Embolic Material Through Clinical Microcatheters

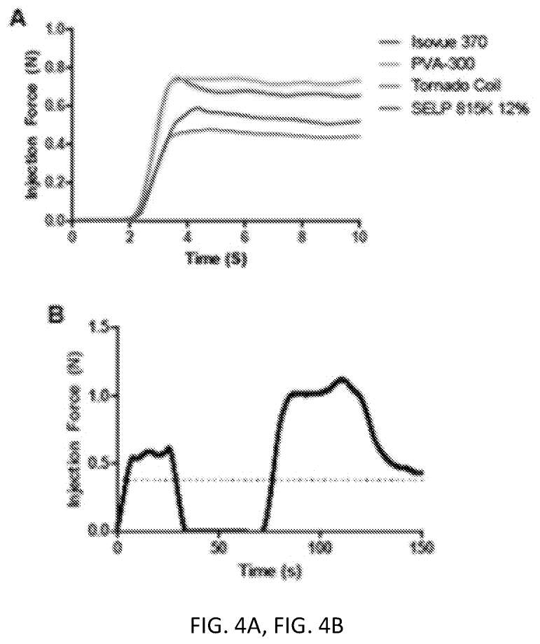

SELP 815K, Isovue 370, PVA 300, and tornado coils were injected through a 2.4 Fr microcatheter to evaluate and compare the force required for injection. A syringe pump equipped with a load cell measured the force used to inject the four different materials. The catheter, submerged in a 37° C. water bath to simulate in vivo administration, was flushed with cold saline between each injection. SELP 815K required a lower injection force than Isovue 370, a clinical contrast agent, and 300-PVA embolic particles (FIG. 4A). During interventional procedures, vasospasms or other events can cause a pause in the administration of treatment. To simulate such occurrences, the injection was paused for 40 s to test if SELP 815K would solidify and clog the catheter. If the injection is paused during the procedure, SELP 815K does begin to transition to a gel within the catheter at 37° C., causing injection to become more difficult (FIG. 4B). However, even after an injection pause of 40 s, SELP does not adhere to the catheter walls and is still injectable as a liquid.

iv. In Vitro Cytotoxicity of SELP Embolic

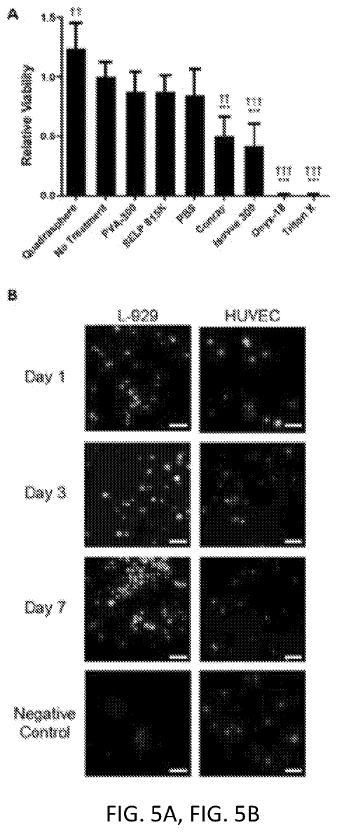

SELP 815K, PBS injection, and PVA-300 embolic particles had similar and minimal impact on L-929 cell viability, indicating that they are not cytotoxic. Conray and Isovue 300 showed substantial depression of cell viability, and Onyx-18® showed similar viability as the negative control (FIG. 5A). Exposure to Onyx® at a 1:10 dilution over a 24 hr period resulted in no viable cells (not shown). Contrast agents and Onyx® exhibited toxicity from prolonged contact with cells under conditions where the opportunity for dilution was limited. L-929 and HUVEC cells incorporated into separate SELP 815K hydrogels showed a high degree of viability. During the 7-day observation period, the L-929 cells multiplied over time within the matrix, while the HUVEC population remained relatively constant (FIG. 5B). These results demonstrate that SELP 815K is cytocompatible.

v. In Vivo Testing of SELP Embolic in a Rabbit Elastase-Induced Aneurysm Model

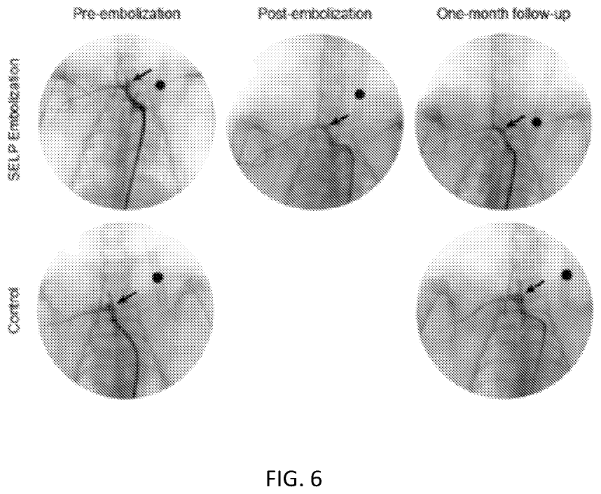

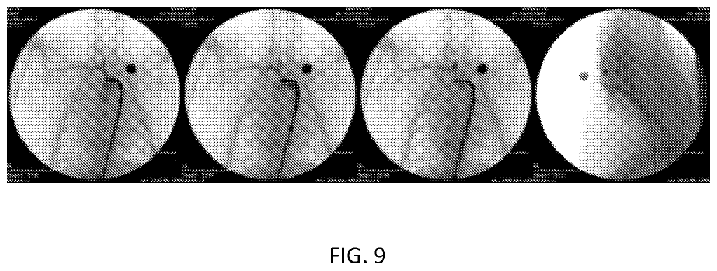

SELP was able to produce effective occlusion in a rabbit model of CA (FIG. 6). Fifteen animals were enrolled in the study and had aneurysms surgically generated. Intake angiography one-month post aneurysm generation showed that the aneurysms formed in the rabbit RCCA had volumes of 46±1 mm³ (mean±st. dev.) (FIG. 9). Ten animals were selected for treatment, and four were selected as controls, based on the intake angiography.

SELP 815K, at 1× to 4× the estimated aneurysm volume, was injected into the aneurysm through a 1.7 Fr catheter by hand without difficulty. This increased injection volume compared to size estimate was to overcome the dilution effects of blood within the aneurysm during embolization. Initially, the balloon catheter blocked the aneurysm for 30 min (n=7), but after establishing procedure, reduction of time to 10 min (n=3) gave proper aneurysm occlusion. No appreciable reduction in function was noted after the decrease in time under balloon protection. Additionally, 1× aneurysm volume injection produced no visible signs of occlusion in the aneurysm after injection (n=1). Increasing the injection volume to 2× the estimated aneurysm volume resulted in 50% occlusion of the aneurysmal sack (n=1). At 3.5-4.0×, the aneurysms were nearly totally occluded, with only minor neck remnants present in a few cases (n=5).

In one case, the entire injection volume, 4×, was released into the bloodstream when the microcatheter slipped out of the aneurysm during the injection of the SELP 815K. Follow-up angiography showed no signs of distal embolization, and the animal showed no signs of distress. A second treatment of the aneurysm was performed.

During the one-month follow-up period, one animal that received SELP embolic suffered hind limb paralysis (3.5× aneurysm volume injection, 30 minutes of balloon protection). Necropsy showed probable trauma to the L5 vertebrae, and there was no indication that this was associated with device administration. The cause of the injury was undetermined. No other animal showed any adverse signs or distress in the 30 days follow-up.

Angiography 30 days post embolization showed continued embolization in most cases. In 2 of the 8 animals, where effective embolization with SELP was achieved under acute angiography, contrast entered slowly into the aneurysmal sac. However, the flow was slow and lethargic. Gross examination of the aneurysms at necropsy showed that the SELP gel was still present within the aneurysm but that the aneurysm had expanded around the gel or the gel had shrunk and become delaminated from the aneurysm wall. However, in both cases, the embolic was still occluding the majority of the aneurysm's volume.

vi. Histological and Gross Examination of SELP Embolization

Gross anatomical examination, performed by a veterinarian upon necropsy, showed no signs of adverse reaction 30 days after implantation for any of the animals that received SELP embolic treatment (n=9). Observation of yellow discoloration on the RCCA on both the untreated control animals and animals embolized indicates that it is the result of vessel ligation and exposure to elastase. In animals embolized with SELP, the aneurysm remained swollen post mortem. However, in the control animals, the aneurysm deflated due to the lack of internal support or pressure. No signs of distal embolization were observed even in animals where the SELP embolic escaped the aneurysmal sac during administration either due to dilution of the material causing a failure to gel or due to improper positioning of the catheter during injection of 4×SELP 815K (n=1).

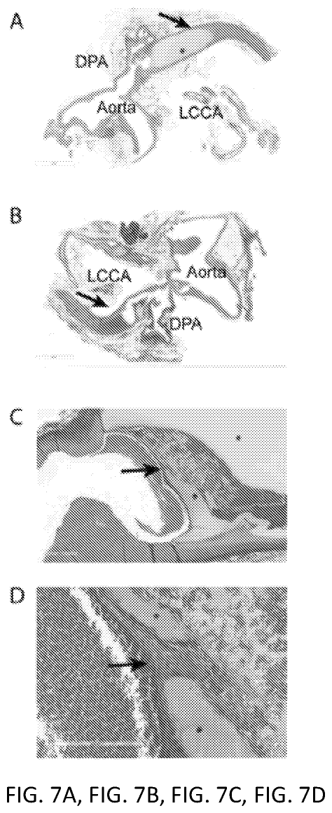

Histologic evaluation showed SELP 815K forming robust gel structure confined to the inner lumen of the aneurysm (FIG. 7), consistent with successful embolization in 8 of 9 cases. SELP stained pale blue on Masson's trichrome special stain and appeared as homogenous nonreflectile and non-polarizable pink foreign material on the evaluated Hematoxylin and Eosin (H and E) stained sections. Smaller fragments of the same foreign material were also noted within the granulation tissue of the aneurysmal sac. There was a mild amount of associated foreign body giant cell reaction and admixed inflammatory response composed of histocytes, eosinophils, and lymphocytes. SELP was not identified outside the aneurysmal sac in the surrounding tissues or vessels histologically. Focal neointimal growth was noted at the neck of the aneurysms in cases with SELP embolization. The amount of new vascular endothelium observed one month after embolization varied between the animals. Histology from the 4^{th} animal treated, with 3.7×SELP volume administered, show regrowth across the entire aneurysm neck (FIG. 7). Due to the use of less than 4×SELP embolic administration, there is a neck remnant in the aneurysm. Additionally, both controls and experimental samples showed variable amounts of granulation tissue, mostly within the aneurysmal sac with hemosiderin deposition, hemorrhage, and organizing thrombus. A polarizable foreign material, most likely representing suture, was noted in only one case.

vii. Histological Evaluation of Downstream Tissues for Signs of Off-Target Embolization

Microscopic examination of the skeletal muscle obtained from the right forelimb revealed only focal inflammation in two samples, indicating the possibility of focal minimal and nonspecific skeletal muscle injury. The examined brain tissue showed no diagnostic abnormalities. Splenic tissue showed focal hemosiderin only without other significant histologic alterations. There was no identified tissue necrosis, significant fibrosis, intravascular embolic material, or prominent inflammatory infiltrates.

4. Discussion

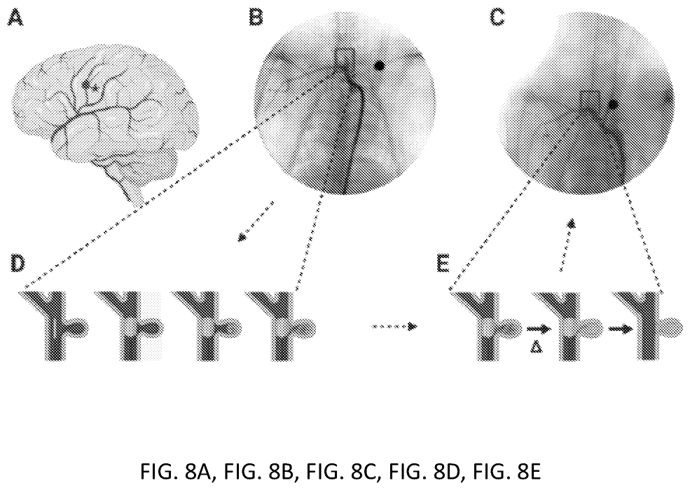

SELP 815K takes advantage of the intrinsic benefits of liquid embolic systems, including complete filling of the aneurysmal sac, injectability through the smallest of catheters, not requiring long-term antiplatelet therapy, and creating occlusion independent of thrombus. Rationale for this work is depicted in FIG. 8. SELP demonstrated the potential to meet all of these features in an in vivo rabbit model of CA.

The peak modulus of embolic gels should be at least that of the thrombi generated from coiled-based embolization devices. Fibrin clots have rheological storage moduli (G′) ranging from 150-1000 Pa. Materials that have high apparent viscosity within the aneurysm sac are beneficial as long as the material shear thins enough to be injectable via microcatheters. SELP 815K embolic achieved a storage modulus of greater than 1000 Pa within 1.5 min at 37° C. Additionally, the embolic was effectively integrated and deployed using a variety of catheters currently available in the interventionist's armamentarium forming a durable occlusion of the aneurysmal sac in less than 10 min. SELP 815K demonstrated shear thinning behavior, which is advantageous for use in microcatheters. Newtonian fluids experience ˜120 Hz from blood flow past the aneurysm neck and 3000 Hz for 0.5 mL/min injection through a 1.8 Fr microcatheter.

An additional advantage of SELP liquid embolics is their possible use for delivering biotherapeutics to the aneurysm sac. Embolics composed of EVOH or metal are poorly suited for delivering biotherapeutics due to the use of cytotoxic organic solvents and limited surface areas. SELP's aqueous environment is ideal for delivering biotherapeutics. SELP 815K demonstrated viability of loaded cells out to 1 week with no adverse effects observed, opening the door for locally directed embolotherapy with adjuvant cell therapy. Local delivery by SELP enhances the concentration of therapeutics within the aneurysm sac, prevents side effects from occurring in nontarget tissues, and increases the effective duration of treatment. SELP also provides a potential platform for delivering cell therapies selectively to the aneurysmal sac that show promise in improving aneurysm healing. Previous work shows SELP 815K to be compatible with local delivery of therapeutic agents, including stem cells, drugs, biotherapeutics, and gene therapy agents for periods of 28 days or longer in vitro and in vivo. Other liquid to solid transitioning embolics under investigation use chemical crosslinking agents that can produce toxic byproducts, high osmolarity solutions that have cytotoxic effects, or materials that have interfering mechanical properties. The limited surface area on stents and coils limits the number of cells that can be loaded, and cell seeding must be performed immediately prior to the administration, which complicates procedures. SELP can be loaded with cells throughout the entire material, drastically increasing the number of cells that can be delivered during treatment. Administration of cells within SELP localizes them to the aneurysmal sac, shields them from the immune system, and increases their efficacy by providing a support structure. The incorporation of therapeutics into a SELP embolic is a potential avenue for developing CA treatments that combine therapeutic elements with embolization. We envision future work creating bioactive embolic materials from the basic SELP backbone, where functional peptides accelerate endothelialization of the aneurysm neck, tune the mechanical properties of the material, or control the release of therapeutics.

Contrast ingress into the SELP embolized aneurysms was observed in 25% of animals (2 out of 8) after a successful initial embolization for 30 days. The ingress could be due to either growth/stretching of the aneurysm or shrinkage of the SELP embolus. It is unclear from either angiography and histology which of these two events occurred. However, for aneurysms treated with embolic coils, the clinical rate of recanalization has been reported as 25.5%. Further testing is needed to address this issue. In either case, the problem can be addressed through careful design. Adding peptide motifs that bind to endothelial cells, such as RGD integrin-binding domains, into the SELP backbone could help the SELP form intimal contact with the vascular endothelium. The addition of a particle or radiopaque particle, such as Ta, to SELP, will help reduce net volume change of the SELP if the contraction of the SELP matrix is an issue. This strategy has been previously used with dental adhesives to prevent contraction during and after curing. Additionally, SELP embolic could be combined with crosslinking agents to enable chemical bonding to the aneurysmal sac. Past work has shown this to be an effective technique where a robust SELP-tissue interface is needed with no localized toxicities. Additional work to understand and prevent the observed partial restoration of flow into the aneurysmal sacs of some aneurysms embolized with SELP is also needed. Preventing recanalization is key to the future translational potential of the SELP embolic system in the treatment of CA.

SELP did not embolize distal tissues in the event where materials were flushed into systemic circulation due to dilution impairing gelation kinetics. In a previous study, SELP embolic for transarterial embolism was injected at high concentrations and higher volumes into small vessels in a low-pressure liver, with no note of distal embolization in the lungs, the predominant down-stream vascular bed after passage through the liver from hepatic artery access. At concentrations below 2% (wt/wt), SELP 815K does not form a cohesive gel even after 24 hours at 37° C. Assuming a 0.5 mL/min injection rate into an artery flowing at 300 mL/min, reasonable for many high flow areas where aneurysms form, there is a 600× dilution in the SELP concentration right away, placing the material well below its minimum gel concentration and rendering it unable to form occlusive particles. Rapid dilution to less than 2% represents a 100× margin of safety. Further dilution will cause the SELP to form small globular protein structures at concentrations below 2 mg/mL of SELP. The intrinsic limitation for SELP gelation to occur only in areas where the material is maintained at gelling concentrations for sufficient time allows for the material to be safely administered even without being radiopaque, as we demonstrated in this in vivo pilot study.

This study demonstrates a high degree of potential for SELP embolic's use in the treatment of cerebral aneurysms. After developing the protocol with the administration of 4× SELP and the balloon inflated for 10 minutes, complete filling of the aneurysmal sac was observed (FIG. 7). SELP embolic demonstrated regrowth of vascular endothelium one-month post embolization, which is a highly promising indication (FIG. 7). The amount of regrowth of the endothelium varied between animals, and in the 4th study animal, treated with 3.7×SELP embolic, new endothelium regrowth over the complete aneurysmal neck is observed. Due to treatment with less than 4× the aneurysm volume, a small neck remnant is present. The formation of new vascular endothelium over the aneurysm neck would eliminate the chance of recanalization, a vast improvement on current embolization therapies, and the formation of a new vascular endothelium over the aneurysm neck is the ideal endpoint for aneurysm embolization. Current liquid embolic systems use potentially toxic organic solvents or release inflammatory byproducts from their polymerization. In one instance, the SELP embolic was accidentally injected outside the balloon occluded aneurysm directly into the bloodstream. No peripheral or off target embolizations were observed, and no deleterious effects on the animal health and wellbeing were observed during the 30-day observation period. Histological examination of the right forelimb and brain showed no signs of microemboli. SELP embolic is a liquid system that uses aqueous PBS as the liquid phase and solidifies in situ without producing any byproducts. For these reasons, SELP can be used as a next-generation embolic material.

### B. Example 2

1. Introduction

Novel embolic therapies are needed to improve cerebral aneurysm (CA) healing and to reduce the risk of recanalization. Treatments for CA have remained virtually unchanged in the last 14 years in spite of the high morbidity and high probability of severe mental disability associated with this disease. CA is a common vascular malformation, comprised of a bulge in a weakened vessel wall that is present in 3.2% of the general population. While CAs are typically asymptomatic, they can rupture spontaneously and cause severe hemorrhagic stroke. Rupture is fatal in 65% of cases and causes severe disability in over 50% of survivors. CAs are especially difficult to treat due to the risks associated with damaging nearby healthy tissue during the intervention. Current therapies prevent rupture by diverting flow away from the aneurysm either by filling the aneurysmal sac with an embolic material or diverting flow using a stent-like device. However, current treatments often fail due to recanalization in 20-57% of cases for unruptured aneurysms. An ideal treatment for CA would be minimally invasive, reinforce weakened vasculature, reduce shear forces on aneurysmal wall, and facilitate healing.

Embolization and flow diversion are the standards of care to prevent intracranial hemorrhage in high-risk patients. However, metal embolization coils and flow diversion devices need larger catheters for delivery, require anticoagulation, cause artifacts on follow-up imaging via magnetic resonance imaging (MRI) or computed tomography imaging (CT), induce thrombus, and can undergo recanalization. Liquid embolics have the potential to fill an aneurysm completely, but current liquid embolics are challenging to use due to their high viscosity, limited selection of compatible catheters, and dependence on potentially toxic organic solvents. Next-generation liquid embolics will have the advantages of not using potentially toxic organic solvents, low viscosity to allow flow through small-diameter microcatheters, and have the capacity to carry various classes of therapeutics while providing durable embolization of the target lumen.

To create a liquid embolic agent that meets these requirements, a recombinant protein-based polymer with adjustable solubility and mechanical characteristics was used. Such an embolic can block flow to the aneurysmal sac reducing mechanical strain and reinforce weakened vasculature preventing the aneurysm from growing. Protein-based polymers have exquisitely defined structures derived from genetic instructions that define monomer sequences and molecular weight, allowing for the precise tailoring of structure to meet functional requirements. Silk-elastinlike protein polymers (SELP) combine the solubility of mammalian elastin and the strength of silk to create molecules with tunable solubility and mechanical properties. Properties such as gelation rate, mechanical rigidity, and network density are dictated by the ratio and sequence of silk and elastin motifs, polymer length, and concentration in solution. Previously, we demonstrated that two unique SELP constructs, SELP 815K and SELP 47K, when dissolved in phosphate-buffered saline (PBS) remain as injectable solutions at room temperature, are able to pass through catheters without occluding, and still rapidly transition to a solid hydrogel after injection. SELPs have demonstrated in vivo stability for greater than 12 weeks and have shown no evidence of toxicity or excessive inflammation.

Silk-elastinlike protein polymers can be designed safely and effectively to occlude cerebral aneurysms. SELP can be combined with radiopacifying agents to generate an injectable liquid solution that solidifies after injection. The objective of this work is to evaluate the basic physicochemical properties of embolic formulation for use in embolizing CA.

2. Materials and Methods

i. Production of SELP Embolic

SELP 815K, which contains 8 silklike motifs, 15 elastinlike motifs, and 1 lysine-substituted elastinlike motif per monomer repeat, was produced from a recombinant plasmid (FIG. 1). The SELP 815K was purified from the crude biomass and sheared as a 12% (wt %) based on a modified version of what has previously been reported. SELP 815K was produced via expression of the pPT-317-SELP 815K-6 mer plasmid in ECR3 E. coli in a Bioflo™ 115 fermenter (New Brunswick Scientific Co., Edison, N.J.). Starting with 6.0 L of MM50 media, 0.4-0.8 L of inoculum was added to begin production. The fermenter was set to run at 30° C., pH of 6.8, airflow of 8-15 L/min., and agitation rate of 1000 RPM. The fermenter monitored pH and foam level and regulated with ammonium hydroxide and Antifoam 204™, respectively. Once the initial glucose was exhausted, the administration of a 600 g/L glucose and 200 mg/L kanamycin feed solution was initiated at a rate of 150 mL/hr. When the optical density at 600 nm reaches 80-100, SELP expression was induced by heating the culture to 42° C. for 30 min. The temperature was then decreased to 40° C. and the glucose feed reduced to 100 ml/hr. for 8 hrs. At the conclusion of the run, the wet biomass was harvested by cooling the culture to below 15° C. and centrifuging the media at 6800 rcf for 30 min. The amount of wet biomass collected ranged from 916-1474 g. The biomass was stored in a −80° C. freezer until purification. Purification began by thawing the biomass and mechanically lysing the cells using a microfluidics microfluidizer 110M at 10,000 PSI. DNA, cell debris, and other negatively charged impurities were removed via polyethyleneimine precipitation and centrifugation. SELP 815K was then precipitated from the supernatant with ammonium sulfate (AS) and solubilized using concentrated formic acid. After another round of AS precipitation, the polymer was further purified using both cation and anion exchange chromatography. Salt content and fluid volume were reduced at various stages using tangential flow filtration with a 35 kDa molecular weight cut off filter. The polymer was then lyophilized.

While on ice, lyophilized SELP 815K was dissolved in DPBS (Gibco, calcium and magnesium-free) to form a 12 wt/wt % solution to produce the liquid embolic material and then sheared at 15 000 PSI using an Avestin C5 homogenizer (Avstin, Inc., Ottawa, Ontario, Canada) fitted with a 1/16″ stainless steel cooling loop modification and a photochemical reactor enhancement detection (PHRED) UV-C system (Aurora, Inc, New York, N.Y.). The Avestin, with attachments, was operated while covered in ice inside a biosafety cabinet. The solution was sheared and sterilized using the PHRED UV-C system before loading in 3 mL BD syringes, and flash-frozen in liquid nitrogen. The syringes were loaded with ˜1 mL SELP 815K 12 wt/wt % liquid embolic. After flash-freezing, the syringes were loaded in zip lock bags and stored at −80° C. The sheared 12 wt/wt % SELP 815K was then flash-frozen in liquid nitrogen, stored at −80° C., and thawed at room temperature with deionized water just prior to use. Iothalmate (from Conray®, Liebel-Flarsheim Company LLC Raleigh, N.C.), an ionic contrast media, and iodixanol (from Visipaque™, GE Healthcare Inc., Princeton, N.J.), a nonionic contrast, were diluted with DPBS to achieve concentrations of 200 mg I/ml in solution and used in place of buffer to generate 12% (wt/vol) SELP embolic formulations. Micronized Tantalum (Ta) was incorporated into standard SELP embolic by mixing in an appropriate volume and mixing with a positive displacement pipettor, with care taken not to introduce air bubbles.

ii. Rheology

Characterization of SELP embolic temperature response was performed using a TA 550 stress-controlled rheometer (TA Instruments, New Castle, Del.) with a stainless steel 4° 20 mm diameter cone and plate geometry. An oscillatory sweep at 6.283 rad/s and 0.1% strain was performed on the material. The temperature was held at 23° C. for 30 min. before it was increased (10° C./min. ramp) up to 37° C. and held there for 1 hr.

Subsequent rheology to analyze viscosity, gelation kinetics, and mechanical durability of the radiopaque SELP was conducted on a Malvern Kinexus Ultra+ Rheometer (Malvern panalytical Ltd, Egham, Surrey, United Kingdom) with a 2° 20 mm stainless steel cone and plate geometry. An active solvent trap was placed around the periphery of the plate to reduce water loss due to evaporation during procedures.

Viscosity was measured from 1 to 37° C. (5° C./min) using an oscillatory procedure at an angular frequency of 6.283 rad/s. This was immediately followed by a 3-hrs. oscillatory sweep at 37° C. using 0.1% strain and an angular frequency of 6.283 rad/s to monitor gelation kinetics as well as G′ and G″. To assess yield strength, an oscillatory amplitude sweep was conducted at 37° C. from 0.01 to 100% strain. All runs were conducted at least in triplicate.

iii. Evaluation of Radiopacity

An Artis Q fluoroscope (Siemens Healthcare Diagnostics, Inc, Tarrytown, N.Y.) was used to acquire images for assessing the relative radiopacity of materials. Iodixanol contrast was serially diluted by 12.5% intervals from full strength and then loaded into 1.6 mm, 0.86 mm, and 0.58 mm diameter polyethylene tubes. A Tornado® Embolization Coil (Cook Medical LLC., Bloomington, Ind.) and a microcatheter tip (Merit Medical, South Jordan, Utah) were used as references of radiopaque devices. Graded wedges of 6061 Aluminum with steps ranging from 1 to 15 mm in 1 mm increments were used to provide a gradient and allow for quantitative assessment of radiopacity. Images were analyzed with ImageJ (National Institutes of Health, Bethesda, Md.) by taking the mean pixel intensity over the area covered by each sample. Radiopaque SELP embolic was also loaded into polyethylene tubes for assessment.

iv. Tilt Testing Method for Assessing Gelation

To evaluate gelation, 400 uL of SELP 815K was loaded into hermetically sealed vials, then tilted 90° and imaged using a digital camera. The vials were placed in a 37° C. water bath and imaged again after 30 sec., 1 min., 2 min., 3 min., 5 min., 10 min., 30 min., and 60 min.

v. In Vitro Injection Testing

Injection testing was used to assess the injectability of embolic formulations and compare them to clinically used devices as a point of reference. A Harvard Apparatus 22 V008 syringe pump (Harvard Apparatus, Holliston, Mass.) was outfitted with a low profile USB output load cell (Omega Engineering, Karvina, Czech Republic) with recordings of the signal made using the associated Omega Digital Transducer software version 2.3.0. PVA-300 Foam Embolization Particles (Cook Medical LLC., Bloomington, Ind.), a Tornado® Embolization Coil (Cook Medical LLC., Bloomington, Ind.) and Isovue 370 (Bracco Diagnostics Inc., Monroe Township, N.J.) were used as reference for the injection force of various materials. Injections were performed using 1 ml BD syringes at a rate of 0.5 ml/min. through a 2.4 Fr 150 cm long Merit Maestro Microcatheter submerged in a 37° C. water bath. A holder was used to ensure that each catheter was in the correct position. Clinical embolics were prepared according to manufacturer directions and administered through the catheter as described above. Prior to SELP injection, cold saline was flushed through the system. After the complete syringe was injected, a second syringe of cold saline was used to push the remaining SELP embolic from the catheter.

vi. In Vitro Embolization of a Model Aneurysm

In vitro embolization was performed on a simulated internal carotid artery aneurysm in a cerebrovascular controlled flow loop (Vascular Simulations, Inc., Stony Brook, N.Y.). The model was submerged in a water bath and maintained at 37° C. SELP was injected into the aneurysmal sac with a 2.3F 110 cm Maestro® microcatheter while a 3 mm Advocate™ balloon catheter was used to block the aneurysm neck. The catheters were removed after 5 min. and the embolic material was observed. A lubricating simulated blood system was made with PBS, mannitol (100 g/l), glycerol (2.5 g/l), and poloxamer 407 (2.5 g/l). The aneurysm has a neck diameter of 4.5 mm, height of 5.2 mm, and width of 4.7 mm with the feeding artery having a diameter of 3.7 mm. PBS flowed through the model at 300 mL/min. to match normal physiological flow through the ICA, which ranges from 246-317 ml/min. Premixed red dye (McCormick & Company, Inc., Baltimore, Md.) was used to provide visual contrast to validate flow. 0.1 mg FD&C emerald green dye (Spectrum Chemical Manufacturing Corp., Newbrunswick, N.J.) was added to 0.2 ml of SELP embolic immediately prior to embolization to facilitate visualization.

vii. Sterility and Endotoxin Testing

Bacterial endotoxin and product sterility tests were performed at Nelson Labs (West Jordan, Utah). All testing at Nelson Labs was performed in accordance with US FDA good manufacturing practice (GMP) regulations 21 CFR parts 210, 211, and 820. Tests were performed on thawed syringes of SELP 815K to verify the endotoxin level and product sterility of the batch. In accordance with USP<71>, a Sterility and MPN Method Suitability (B/F) test was performed to verify that the liquid embolic did not suppress bacteriostasis or fungistasis.

viii. Cytotoxicity

L-929 and HUVEC cells were cultured and seeded into 96-well plates as described in Section 5.2.8. However, 1% Pen-Strep was added to the media for the study as cells would be cultured for an extended period with multiple media changes. Viability was measured after 24 hrs using a Cell Counting Kit (CCK)-8 assay kit (Dojindo, Kumamoto, Japan) per manufacturer's directions. No treatment and 1% Triton-X were used as positive and negative controls, respectively. DPBS was used as an additional control to account for media dilution. Onyx 18™ (ev3 Inc., Plymouth, Minn.), Quadrasphere® (Merit Medical, South Jordan, Utah), PVA-300 microspheres, Isovue, and Conray 60% were used as reference materials. HUVEC and L-929 cells were gently mixed into freshly thawed SELP embolic. The SELP mixture was then loaded into a tuberculin syringe and incubated at 37° C. The end of the syringe was removed, and the resulting cylinder was sectioned into 20 μl disks and placed in media. Negative control gels were incubated in media with 0.1% Triton X for 30 min. at 37° C. Live/Dead Assay (ThermoFisher Scientific, Waltham, Mass.) was used to stain cells within the gels prior to imaging on an FV1000 Olympus Confocal Microscope using the manufacture's recommended settings.

ix. In Vivo Testing of SELP Embolic in a Rabbit Cerebral Aneurysm Model

Aneurysms were generated in New Zealand White rabbits in the right common carotid artery (RCCA). Briefly, aneurysm generation involves surgically isolating the RCCA, advancing a balloon catheter to the origin of the RCCA through a vascular sheath, inflating the balloon catheter to occlude flow into the RCCA, injecting Porcine Elastase (50 U/ml) into the RCCA, and incubating for 20 min. to induce aneurysm formation. The balloon catheter was then deflated and removed, the vessel was rinsed with saline, the sheath and microcatheter were removed, and the distal RCCA was ligated. Embolization of the aneurysm models was performed 30-31 days after aneurysm creation to allow for the model aneurysm to mature and the animals to recover.

The right femoral artery was surgically accessed, and a 6F radial sheath was inserted into the femoral artery and a bolus injection of heparin administered. A Cordis MPA 6F×100 cm guide catheter was directed under fluoroscopy to near the origin of the RCCA. A Transform® Compliant Occlusion Balloon Catheter (4 mm×10 mm×150 cm) (Stryker, Kalamazoo, Mich.) was placed near the aneurysm neck. An Excelsior® SL-10 microcatheter (150 cm long, 1.7Fr, 0.60 mm) (Stryker, Kalamazoo, Mich.) was placed within the aneurysm. Angiography was performed to measure the aneurysm dimensions, and an ellipsoid model per equation 6.1 (W: width, D: depth, H: height) was used to calculate the volume of SELP injection. The injected volume of SELP was stepped up from 1× the estimated aneurysm volume to 4× the aneurysm volume until >90% filling of the aneurysmal sack was observed on follow-up angiography. Time of balloon occlusion was reduced from 30 min. to 10 min. after through occlusion of the aneurysm was established at the longer timepoint. SELP was then prepared for injection by thawing the syringe in sterile saline. SELP proportional to the aneurysm volume plus the catheter hold up volume was injected. Slight negative pressure was applied to the microcatheter, and then it was retrieved past the occluding balloon. The balloon catheter was then left in place to allow the SELP to solidify. After the balloon catheter was removed, angiography was performed again and the volume of occlusion visually assessed. 30 days after initial embolization, the left femoral artery was accessed as described above and a guide catheter was used to deliver contrast to perform angiography.

V=(π×W×D×H)/6 Equation 6.1

x. Gross Evaluation of Embolization Via Necropsy

Macroscopic examination of the animals was performed by a veterinarian. The aneurysm and surrounding vasculature were isolated, inspected, and photographed. Other tissues were inspected and evaluated for lesions or any other signs of adverse events. The right forelimb, brain, and the aneurysm with surrounding vasculature were collected and fixed in formalin 10%.

xi. Histology

The aneurysm and surrounding vasculature were processed into a single block to provide anatomical context to the histology. At least two sections of the supraspinatus and suprapliaris muscles from the right forelimb and two sections from the brain for each animal were obtained and processed for the evaluation of off-target effects, as these tissues are down-stream from the site of embolization. Each set of tissue sections was embedded in paraffin, sectioned into 5 um slices, and stained with hematoxylin and eosin (H&E) by the research histology core. The aneurysms and associated vasculature were additionally stained using Masson's trichrome.

xii. Statistics

Data were recorded, organized, and processed using Excel® (Microsoft, Redmond, Wash.). GraphPad Prism 5.0 was used to perform statistical analysis. All numerical data in this manuscript were assumed to be parametric in nature. One-way analysis of variance (ANOVA) with a post-hoc Bonferroni multiple comparison test of data sets with 3 or more groups. A p-value of less than 0.05 was used as the threshold to ascribe statistical significance to a result.

3. Results

i. Temperature Responsiveness of SELP

SELP 815K self-assembly responds dynamically to temperature. Elevation from room temperature (23° C.) to 37° C. initiated a rapid shift from a liquid-like state to a solid gel (FIG. 2). The growing separation between the storage and loss modulus is indicative of this transition to increasing stiff materials. The change in storage modulus was over 5.5 logs in magnitude, which is indicatory of adhesive potential.

ii. Incorporation of Radiopacifying Agents into SELP Embolic

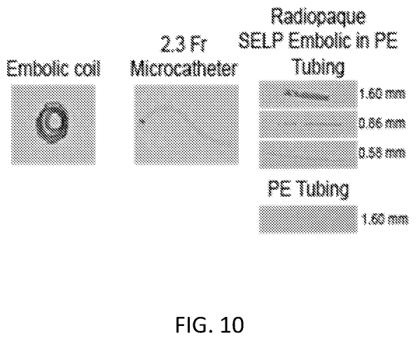

The addition of radiopaque materials to the solution phase of SELP embolic allows for fluoroscopic visualization (FIG. 10). Incorporation into SELP embolic does not meaningfully impact radiopacity. The addition of 200 mg I/ml incorporated from iodinated contrasts is sufficient to enable ready visualization of SELP embolic through neuro-interventional microcatheters during delivery. This was assessed using the degree of contrast of clinically used microcatheters with radiopaque plastic tips as baseline and then assessing the contrast value between that tip and background using the average pixel intensity.

iii. Viscoelastic Properties of Radiopaque SELP Embolic

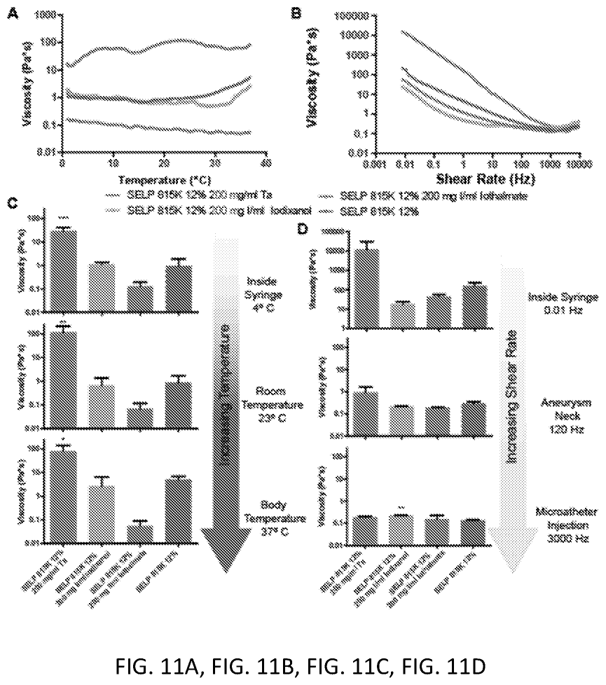

The incorporation of radiopacifying elements into SELP embolic has a dramatic impact on its viscosity profile. Addition of Ta microparticles, as is used in Onyx®, drastically elevates the viscosity of the system at all temperatures and under low shear conditions (FIGS. 11A and 11B). As the SELP network forms with rising temperatures, the viscosity increases. This is further exacerbated beyond simple additive effects by Ta particles creating increased drag within the solution (FIG. 11C). This relationship is confirmed by analyzing shear rate sweep of the materials. All SELP embolic formulations show shear thinning, but the SELP embolic with Ta microparticles has the greatest degree of shear-thinning behavior. As the shear rate increases, intermolecular interactions between SELPs are exceeded by the shear stress and subsequently break down. The SELP embolic with Ta powder demonstrated a higher degree of shear-thinning than any of the other formulations. Newtonian fluids will experience a 3000 Hz shear rate during a 0.5 ml/min. injection through a 1.8Fr (0.60 mm) diameter catheter. All SELP embolics had viscosities that allowed easy injection by hand under these conditions. A material that has a higher apparent viscosity within the aneurysm sac is actually beneficial as long as the material shear-thins enough to be injectable. SELP with either iothalamate or iodixanol had decreased shear-thinning behavior compared to SELP alone, indicating reduced intermolecular polymer interactions. The incorporation of contrast materials to SELP embolic had a pronounced effect on viscoelastic properties.

iv. Test Injections of SELP Embolic Material Through Clinical Microcatheters

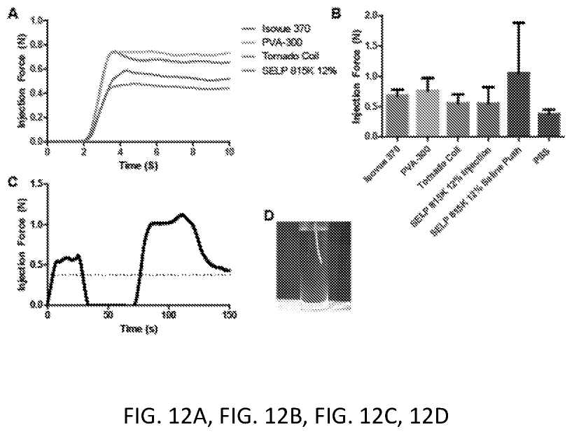

SELP embolic required lower injection force than Isovue 370, a thick clinical contrast agent, or 300-PVA embolic particles at a 0.5 ml/min. injection rate (FIG. 12A). This indicates that SELP embolic is injectable under simulated clinical conditions. SELP embolic does begin to set within the catheter, causing injection to become more difficult (FIGS. 12B and 12C). However, SELP is still injectable and emerges as a liquid form in the catheter even after a paused injection. This allows the SELP to flow and conform intimately with the sac of the aneurysm and produce a thorough occlusion (FIG. 12D). Test injections with viscosity 5000 cP silicone oil standards could not be injected through the microcatheter and stalled the motor on the syringe pump. Injections of 1000 cP silicone oil in a 1.0 mL syringe required 34±6 N for injection through the microcatheter, which far exceeds the force of clinically used systems (FIG. 12B). SELP embolic was easily injectable under simulated clinical conditions.

v. Gelation Kinetics and Mechanical Strength of Radiopaque SELP

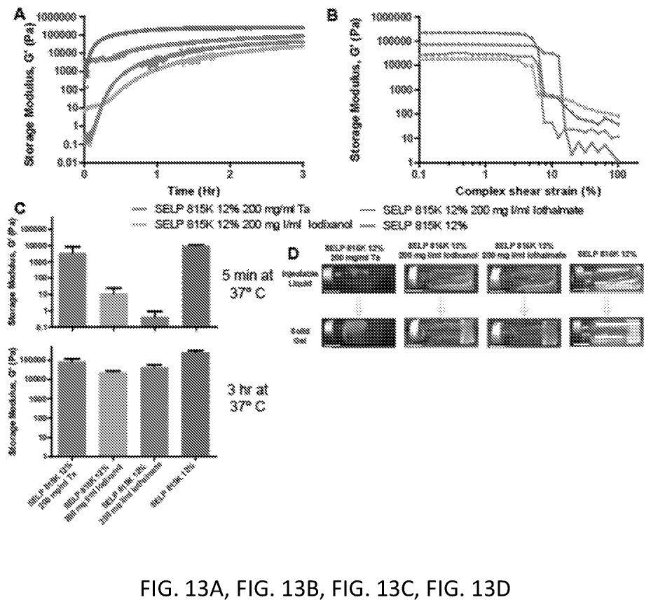

SELP 815K without any additional radiopacifying agents exhibited the greatest rate of gel formation (slope of line in FIG. 13A). SELP with tantalum had a higher initial storage modulus but did not see the same rise in viscosity in that initial time period. The SELP with iodixanol was thicker initially but was passed by the SELP loaded with iothalamate. This indicates that while iothalamate interferes with intermolecular interactions, it does not prevent the formation of the crosslinks among silk units in the gel. The addition of Ta to SELP increased the gel's ability to dissipate energy and thus have a greater capacity to withstand shear strain (FIG. 13B). All of the radiopaque materials had demonstrated the capacity to gel; however, the duration needed to achieve a robust gel varied (FIG. 13C). SELP embolic was able to pass the tilt test by min. 3. SELP with Ta was more viscous and was able to pass the tilt test by min. 2. SELP loaded with iodine-based contrast took between 15 and 30 min. to set to the point where it would pass the tilt test (FIG. 13D).

vi. In Vitro Cytotoxicity of SELP Embolic

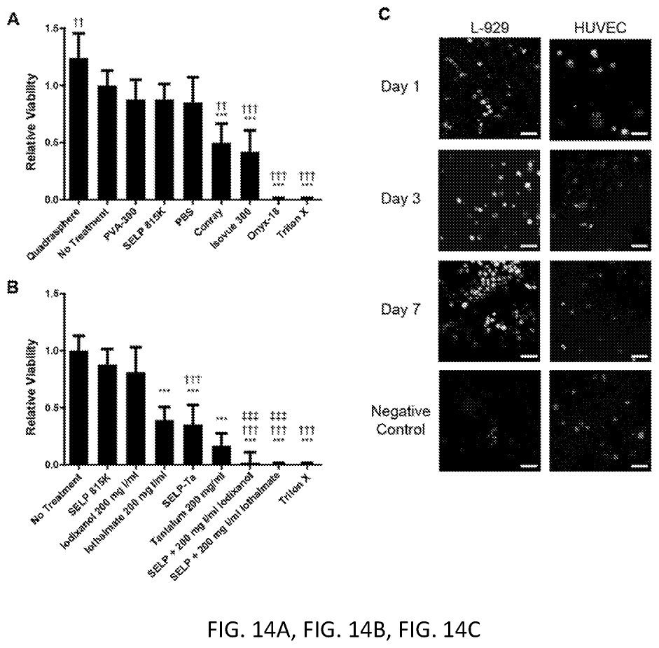

SELP embolic had a similar effect on L-929 cell viability as either a PBS injection or PVA-300 embolic particles. It was less toxic to the cells than either Conray, Isovue 300, or Onyx-18 (FIG. 14A). Onyx at a 1:10 dilution over a 24-hr. period essentially killed all of the cells. Incorporation of organic iodine or tantalum into the SELP embolic increased its cytotoxicity. For iodinated contrasts, in particular, incorporation into SELP resulted in a material that was significantly more toxic than either contrast agent or SELP alone (FIG. 14B). However, the incorporation of Ta into SELP seemed to ameliorate some of its toxicity. L-929 and HUVEC cells showed a high degree of viability after incorporation into SELP. While the L-929 cells were observed to multiply over time within the matrix, the HUVEC population remained consistent over the 7-day observation period (FIG. 14C). Concerns over impaired mechanical function and increased cytotoxicity led us to pursue the standard SELP embolic for in vivo validation over any of the radiopaque formulations tested.

vii. Production of Clinical-Grade SELP Embolic

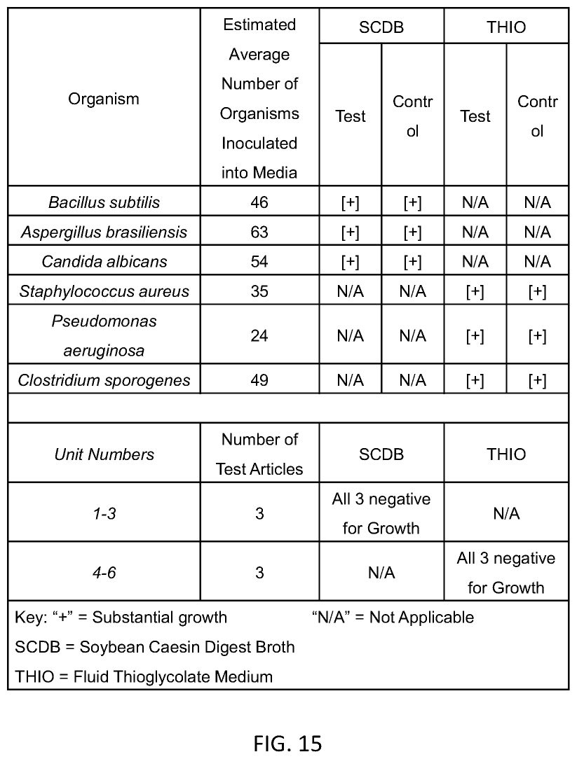

The suitability of the sterility test for SELP 815K 12 wt % liquid embolic was determined using 6 test organisms in two different media types. Growth in the bottles containing the liquid embolic test product was compared to the positive controls (FIG. 15). The test shows that the liquid embolic test product is not inhibitory to microbial growth. Test results for the suitability study are described in Table 6.1. Product sterility test was negative for growth in both soybean casein digest broth and fluid thioglycolate medium (FIG. 15), and bacterial endotoxin level was measured at 0.0425 EU/mL for SELP 815K liquid embolic, which is within FDA guidelines for neuro-interventional devices.

viii. In Vitro Embolization of a Model Human Aneurysm

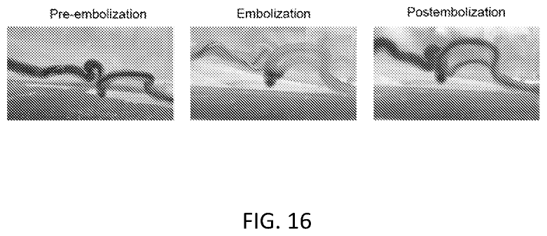

SELP was injectable via microcatheter and able to produce an effective occlusion in a model aneurysm. The balloon catheter was able to maintain the SELP embolic within the aneurysm and allow the material to solidify and produce an effective occlusion of the aneurysm sac (FIG. 16). If the microcatheter was left in place during gelation, it could be easily removed without damaging the gels but did leave a cylindrical void in the gel. Removing the catheter immediately after injection resolved this issue. SELP gel was not adhesive to the surface of the catheter but remained in place after administration due to becoming interlocked with the aneurysm.

ix. In Vivo Testing of SELP Embolic in a Rabbit Elastase-Induced Aneurysm Model

SELP was able to produce effective occlusion in a rabbit model of CA (FIG. 6). Angiography showed that the aneurysms formed were between 6-10 mm deep with depth and width varying from 3-5 mm, with volumes 46±1 mm3 (mean±st. dev.). Test injections of contrast into the aneurysm showed the inflation of the balloon catheter and injections up to 4× the angiographically estimated volume of the aneurysm without any visibly evident release of contrast. SELP embolic at 1× to 4× the estimated aneurysm volume was injected into the aneurysm through a 1.7 Fr catheter by hand without difficulty. The balloon catheter was left in place for either 10 min. (n=3) or 30 min. (n=7). This increased volume compared to size estimate was to overcome the dilution effects of blood within the aneurysm during embolization. In one case, the microcatheter slipped out of the aneurysm during deployment, and the entire volume was released into the bloodstream and diluted. Follow-up angiography showed no signs of distal embolization and the animal showed no signs of distress. Additionally, 1× aneurysm volume injection produced no visible signs of occlusion in the aneurysm after injection (n=1). Increasing the injection volume to 2× the estimated aneurysm volume resulted in 50% occlusion of the aneurysmal sack (n=1). At 3.5-4.0×, the aneurysms were nearly totally occluded with only minor neck remnants present in a few cases (n=5). Once injection volume was established using 30 min. of balloon protection, the time under protection was dropped to 10 min. (n=3). No appreciable reduction in function was noted after the decrease in time under balloon protection.

During the 1-month follow-up period, one animal that received SELP embolic suffered hind limb paralysis (3.5× aneurysm volume injection, 30 min. of balloon protection). However, necropsy showed probable trauma to the L5 vertebrae, and it was determined that this was not associated with device administration. The cause of the trauma was undetermined. No other animal showed any negative signs or distress in the 30 days following the procedure.

Angiography 30 days post-embolization showed continued embolization in most cases. In 2 of the 8 animals, where effective embolization with SELP was achieved under acute angiography, contrast entered slowly into the aneurysmal sac. However, the flow was slow and lethargic. Gross examination of the aneurysms at necropsy showed that the SELP gel was still present within the aneurysm but that the aneurysm had expanded around the gel or the gel had shrunk and become delaminated from the aneurysm wall. However, in both cases, the embolic was still occluding the majority of the aneurysm's volume.

x. Histological and Gross Examination of SELP Embolization

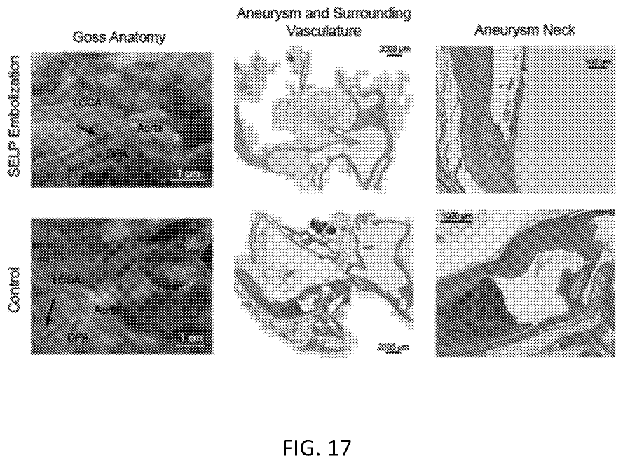

Gross anatomical examination showed no signs of adverse reaction 30 days after implantation for any of the animals that received SELP embolic treatment (n=9) (FIG. 17). A yellow discoloration was observed on the RCCA on both the untreated control animals and animals embolized, indicating that it was the result of vessel ligation and exposure to elastase. In animals embolized with SELP, the aneurysm remained swollen post mortem. However, in the control animals, the aneurysm deflated due to the lack of internal support or pressure. No signs of distal embolization were observed even in animals where the SELP embolic escaped the aneurysmal sac during administration either due to dilution of the material causing a failure to gel (1× aneurysm volume injection n=1) or due to improper positioning of the catheter during injection (n=1)

Histology showed that SELP formed robust gel structure that conformed to the inner lumen of the aneurysm (FIG. 17). SELP is stained with a characteristic pale blue by Masson's trichrome. Neointimal growth was observed over the neck of the SELP lumen interface, which is a strong indication of successful embolization. SELP was observed in the granulation tissue within the RCCA above the aneurysm sac, indicating that SELP was sufficiently low in viscosity to penetrate the spongy tissue formed within the RCCA after aneurysm creation. Foreign body giant cells were observed around these pockets of SELP within the granulation tissue. SELP was not observed in any other portion of the surrounding tissues. In some samples, small pockets of red blood cells were found trapped between SELP and the aneurysm wall, indicating that small zones of entrapped blood were cut off by embolization. Thrombus was observed in the control aneurysms, but not on angiography, indicating that the clot likely formed during necropsy (FIG. 17).

xi. Histological Evaluation of Downstream Tissues for Signs of Off-Target Embolization

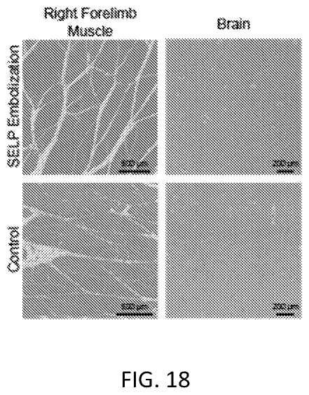

In neither the right forelimb or brain were there any signs of distal embolization or toxic events (FIG. 18). This was true even in animals where procedural errors resulted in SELP being flushed out of the aneurysmal sack and into circulation. This is a surprising observation, as it is expected that SELP would produce occlusive events. However, this was not found to be the case.

4. Discussion

SELP embolic takes advantage of the intrinsic benefits of liquid embolic systems including complete filling of the aneurysmal sac, injectability through the smallest of catheters, not requiring long-term antiplatelet therapy, and creating occlusion independent of thrombus. SELP demonstrated the potential in an in vivo model of CA to meet all of these features.

The peak modulus of embolic gels should be at least that of the thrombi generated from coiled-based embolization devices. Fibrin clots have rheological storage moduli (G′) ranging from 150-1000 Pa. All of the SELP embolics tested in this chapter have far exceeded this requirement for strength. SELP embolic achieved a storage modulus of greater than 1000 Pa within 1.5 min. at 37° C. Additionally, the embolic was effectively integrated and deployed using a variety of catheters currently available in the interventionist's armamentarium. An additional advantage of SELP liquid embolics is their possible use for delivering biotherapeutics to the aneurysm sac. Embolics composed of EVOH or metal are poorly suited for delivering biotherapeutics due to limited surface areas and use of cytotoxic organic solvents, whereas SELPs aqueous environment is ideal for delivering biotherapeutics. SELP embolic demonstrated viability of loaded cells out to 1 week with no adverse effects observed. Local delivery by SELP enhances the concentration of therapeutics within the aneurysm sac, prevents side effects from occurring in nontarget tissues, and increases the effective duration of treatment. SELP also provides a potential platform for delivering cell therapies selectively to the aneurysmal sac that show promise in improving aneurysm healing. Other liquid to solid transitioning embolics under investigation use chemical crosslinking agents that can produce toxic byproducts, use high osmolarity solutions that have cytotoxic effects, or have interfering mechanical properties. The limited surface area on stents and coils limits the numbers of cells that can be loaded and cell seeding must be performed immediately prior to the administration, which complicates procedures. SELP can be loaded with cells throughout the entire material, drastically increasing the number of cells that can be delivered during treatment. Administration of cells within SELP localizes them to the aneurysmal sac, shields them from the immune system, and increases their efficacy by providing a support structure.

Incorporation of contrast agents reduced SELP biocompatibility. This is due to the intrinsic toxicity of the contrast material. However, SELP with contrast was still less cytotoxic than Onyx-18. While no precise values are reported in the literature for either the cytotoxicity or hemolytic potency for Onyx®, the United States Food and Drug Administration Humanitarian Use Device Exemption summary reports that test results for Onyx's cytotoxicity were grade 4 (severe) at a 1:1 dilution and grade 3 (moderate) at a 1:2 dilution. So this type of toxicity observed in cell culture is known. In the body, the high volume of dilution and convective clearance by blood flow quickly dilutes the dimethyl sulfoxide that forms the liquid component of the Onyx embolic. During cell culture studies, there is a limited volume of media and the duration of exposure is much higher than would be expected in vivo. The same scenario likely holds true for the radiopaque SELP formulations that used organically bound iodine. The observed increases in toxicity from the contrast were also probably due to contrast being held in close proximity to the cells rather than diffusing uniformly throughout the media.

Additionally, SELP embolic demonstrated the ability to incorporate cells in this study, opening the door for locally directed embolotherapy with adjuvant cell therapy. Previously, SELPs have been shown to be compatible for local delivery of therapeutic agents including stem cells, drugs, biotherapeutics, and gene therapy agents for periods of 28 days or longer in vitro and in vivo. The incorporation of therapeutics into a SELP embolic can be used for developing CA treatments that combine therapeutic elements with embolization.

Contrast ingress into the SELP embolized aneurysms was observed in 25% of animals (2 out of 8) after an apparently successful initial embolization for 30 days. This could be due to either growth/stretching of the aneurysm or shrinkage of the SELP embolus. It is unclear from either angiography or histology which of these two events occurred. However, clinically the rate of recanalization has been reported as 25.5% for aneurysm treated with embolic coils. Adding peptide motifs that bind to endothelial cells, such as RGD integrin-binding domains, into the SELP backbone could help the SELP form intimal contact with the vascular endothelium. Addition of a particle or radiopaque particle, such as Ta, to SELP will help reduce net volume change of the SELP, if the contraction of the SELP matrix is an issue. This strategy has been previously used with dental adhesives to prevent contraction during and after curing. Additionally, SELP embolic could be combined with crosslinking agents to enable chemical bonding to the aneurysmal sac. Past work has shown this to be an effective technique where a robust SELP-tissue interface is needed with no localized toxicities. Additional work to understand and prevent the observed restoration of flow into the aneurysmal sacs of some aneurysms embolized with SELP is also needed. Preventing recanalization is key to the future translational potential of the SELP embolic system.

SELP did not embolize distal tissues in the event where materials were flushed into systemic circulation due to dilution impairing gelation kinetics. In a previous study, SELP embolic for TAE was injected at high concentrations and higher volumes into small vessels in a low-pressure liver. There was also no note of distal embolization in the lungs, the predominant down-stream vascular bed after passage through the liver from hepatic artery access. SELP 815K below 2% (wt/wt) does not form a cohesive gel even after 24 hrs. at 37° C. Assuming a 0.5 ml/min. injection rate into an artery flowing at 300 ml/min., reasonable for many high flow areas where aneurysms form, there is a 600× dilution in the SELP concentration right away, placing the material well below its minimum gel concentration and rendering it unable to form occlusive particles. Rapid dilution to less than 2% represents a 100× margin of safety. Further dilution will cause the SELP to form small globular protein structures at concentrations below 2 mg/ml of SELP. The intrinsic limitation for SELP gelation to only areas where it is maintained at gelling concentrations for sufficient time allows for the material to be safely administered even without being radiopaque.

SELP embolic demonstrated regrowth of vascular endothelium 1-month post-embolization which is a highly promising indication. Formation of a new vascular endothelium over the aneurysm neck is the ideal endpoint for aneurysm embolization. Current liquid embolic systems use potentially toxic organic solvents or release inflammatory byproducts from their polymerization. Additionally, SELP embolic is a liquid system that used saline as the liquid phase and solidified in situ without producing any byproducts. For these reasons, SELP is a next-generation embolic material.

5. Conclusions

A SELP embolic was produced using SELP 815K and was successfully deployed as an embolic for treating a simulated CA in a fluidic model of human aneurysm and in vivo using an elastase-induced aneurysm model in rabbits. The liquid embolic was able to be injected through a microcatheter and achieves durable gelation that is capable of occluding blood flow to the aneurysmal sac.

Those skilled in the art will recognize, or be able to ascertain using no more than routine experimentation, many equivalents to the specific embodiments of the method and compositions described herein. Such equivalents are intended to be encompassed by the following claims.

### C. Example 3

1. Introduction

Distinguishing tumor margins from normal tissue in endoscopic surgery is challenging, as surgeons heavily rely on palpation and visual inspection of the tissue. This process is especially problematic for endoscopic surgery in the sinonasal cavity, as tumor margins can only be assessed via an endoscope. Maintaining good visualization of the surgical field and being able to identify normal tissue versus the tumor is paramount for safe and successful oncological surgery. Endoscopic surgical resection of hypervascular tumors can be challenging due to reduced visibility from bleeding and inability to palpate tissue, leading to difficulties in identifying normal tissue from tumor tissue and increasing the risk of surgical complications and suboptimal gross tumor resection. These challenges are compounded during the resection of hypervascular tumors in the sinonasal cavity, where excessive intraoperative bleeding can rapidly obscure the visual field due to the limited space and ability to access the tumor in this unique surgical corridor. Juvenile nasopharyngeal angiofibroma (JNA) is particularly difficult to remove, due to their location in the sphenopalatine foremen and proximity to critical structures such as the trigeminal nerve, internal and external carotid, optic nerve, orbit, and the brain. As surgery is currently the most common and effective form of treatment for JNAs, clear and defined margins are critical for achieving optimal outcomes. The development of new methods that can improve intraoperative visualization by reducing bleeding while enhancing demarcation of tumor margins could greatly improve safety and oncologic outcomes in these complex cases.

Fluorescence-based image-guided surgery has shown great potential to intraoperatively detect malignant tissue in endoscopic and robotic surgeries and distinguish tumor margins. Near-infrared (NIR) imaging with fluorescent NIR contrast agents utilize wavelengths in the range of 700-900 nm. NIR imaging minimizes background autofluorescence and allows for the greatest transmission of light within tissues. However, rapid dilution after administration and short circulation time result in low accumulation of dyes within the desired tissues. Embolization provides a unique opportunity to overcome both of these shortcomings, by locally delivering a higher concentration of a fluorescent dye, thereby reducing its clearance from the tumor by occluding blood flow.

Pre-surgical embolization is currently practiced for a variety of tumors in the head and neck in order to reduce intraoperative bleeding during surgical resection. Reduced intraoperative bleeding can help decrease operative time, improve visualization of the surgical field, decrease the risk of surgical complications in adjacent tissues, and decrease the risk of tumor recurrence. Current embolic materials are ill-suited for fluorescent marking due to poor tumor penetration and incompatibility with clinically approved dyes. Particle-based clinical embolics (i.e., microspheres and gelatin foams) can efficiently block tumor blood supply, but these materials fail to deeply penetrate the tumor vasculature. Liquid embolic agents, such as acrylic glues (Truefil™), can only penetrate blood vessels to a depth of 0.5 mm and only spontaneously solidify when polar or charged materials are added. An ideal embolic agent for pre-surgical embolization should be capable of: 1) delivering a marker to tumors, 2) deeply penetrating into and occlude vasculature, and 3) releasing the majority of its payload in accordance with surgical procedural timing.

Indocyanine green (ICG) has shown promise in head and neck surgical procedures for a variety of cancers. ICG binds avidly to albumin and other globular proteins that naturally extravasate into tissue at the capillary level. ICG accumulates preferentially in tumor tissue due to poor lymphatic recycling of albumin and other blood proteins compared with healthy tissues. This difference in clearance can create a well-defined boundary that corresponds to tumor margins. Rapid dilution after intravenous administration and rapid clearance by the liver, half-life of only 3-5 min., limit ICG's ability to accumulate in tumors and successfully demarcate tumor margins. Only approximately 0.05% of an ICG dose typically remains within the tumor by the time of surgery. This challenge could be overcome by locally delivering ICG and restricting blood flow within a tumor.

Silk-elastinlike protein (SELP)-based embolics have the potential to locally deliver ICG while achieving effective embolization. SELPs are genetically engineered protein-based polymers that combine the temperature-responsive solubility of elastin and the physical strength of silk. The ability to control SELPs at a molecular level allows the precise tailoring of protein structure to function in a predictable and exquisitely tunable fashion. SELPs dissolved in saline are highly biocompatible and have mechanical properties for use as an in situ gelling embolic. SELPs represent an innovative solution to overcome the shortcomings of current clinical tools for embolizing hypervascular tumors. These embolics can deeply penetrate the tumor before rapidly transitioning to form a solid gel, use a biocompatible aqueous solution, and can carry up to 50 mg/mL of loaded compounds. Described herein is the development of a dual-function SELP-based embolization-visualization system that can reduce intraoperative bleeding, while simultaneously delivering ICG to fluorescently demarcate tumor margins. We characterized the biophysical properties of SELP in response to ICG incorporation and penetration efficiency in phantom agar tissues. The dual-functionality of the SELP-ICG system was then evaluated in a microfluidic model of tumor vasculature. To assess the biocompatibility of this new system, the viability of model mammalian cell lines in response to SELP-ICG incubation was tested.

2. Materials and Methods

i. Materials

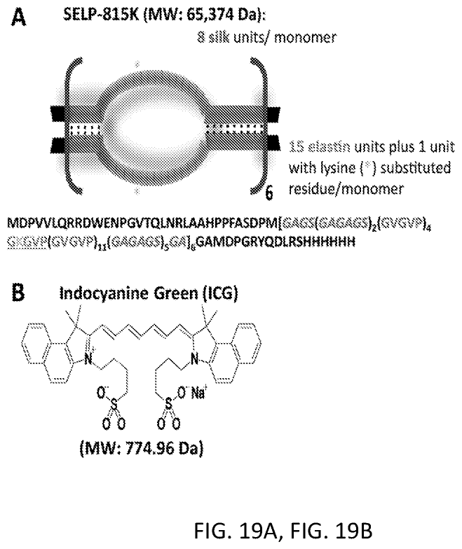

SELP 815K was expressed in Escherichia coli and purified, characterized, and shear-processed as previously described (FIG. 19A). ICG sodium salt (see FIG. 19 B) was obtained from Sigma Aldrich (St. Louis, Mo.). Dulbecco's Phosphate Buffered Saline (PBS), agar, Dulbecco's Modified Eagle Medium/Nutrient Mixture F-12 (DMEM:F12), TrypLE™ Express Enzyme with no phenol red, trypan blue, and Fetal Bovine Serum (FBS) were obtained from ThermoFisher Scientific (Waltham, Mass.). Triton X, sodium azide, Endothelial Cell Growth Medium (ECGM), and bovine serum albumin (BSA) were obtained from Sigma Aldrich (St. Louis, Mo.). FD&C red dyes 40 and 3 were obtained in a premixed solution (McCormick, Hunt Valley, Md.) to serve as visual indicators. L-929, murine fibroblasts, and human umbilical vein endothelial cells (HUVECs) were obtained from the American Type Culture Collection (ATCC) (Manassas, Va.).

ii. Effect of ICG on SELP Hydrogels

To evaluate the effect of ICG on the swelling behavior of SELP hydrogels, frozen SELP 815K 12% (wt/wt) was thawed, mixed with 0, 0.1, 1.0, 5.0, and 10.0 mg/mL ICG, and then incubated for 12 hrs. at 37° C. in tuberculin syringes. These concentrations spanned the range above and below concentrations used clinically for ICG injections. The end of the syringe was removed, and the SELP-ICG mixture was cut into 20±1 μL cylindrical samples (˜3.5 mm in diameter, ˜2 mm in height) and weighed. These were placed into 1.0 mL of PBS and incubated for 2 weeks at 37° C. Samples of SELP with and without 0.5 mg/mL ICG were flash-frozen in liquid nitrogen and lyophilized at −50° C. and <0.06 mbar for 4 days on Labconco lyophilizer (Kansas City, Mo.). Free ICG and ICG incorporation into the gels were quantified using absorbance detection at 780 nm with a SpectraMax M2 spectrophotometer (Molecular Devices, Sunnyvale, Calif.). The swelling ratios and soluble fractions were calculated. Scanning electron microscopy (SEM) was performed on an FEI Quanta 600F (ThermoFisher Scientific, Waltham, Mass.) as previously described to evaluate the possible effects of ICG incorporation on SELP microstructure morphology.

iii. ICG Release from SELP

A release study was conducted to determine the effect of ICG concentration on its release profiles from the hydrogels. SELP-ICG was loaded with concentrations of dye appropriate for imaging after local delivery (0.005, 0.05, and 0.5 mg/mL) by directly mixing the powdered dye into the polymer solution. Beginning with 20 μL of SELP 815K 12% (wt/wt) (n=5), a concentration that has previously demonstrated embolic potential, was injected into a 1 mL vial through a 30 g needle with a chilled Hamilton syringe. SELP was then incubated at 37° C. for 12 hrs. To begin the release study, 1 mL of prewarmed (37° C.) PBS supplemented with 50 mg/mL of bovine serum albumin (BSA) was added to the samples. As a control for ICG stability, 1 mg/mL of ICG dissolved in release media was used and treated identically to samples. Prior to injection, the release media was prewarmed to 37° C. At designated timepoints (0, 0.25, 0.5, 1, 3, 6, 12, 24, 36, and 48 hrs.), 100 μL of media was removed and replaced from each vial, added to a 96-well plate, and assayed at 780 nm on a SpectraMax M2 spectrophotometer. To analyze the release profile of ICG from the SELP hydrogels, the data were fit to the Korsmeyer-Peppas model (Equation 5.1).

Mₜ/M_{∞}=ktⁿ (5.1)

iv. Viscoelastic Properties of SELP Embolic Loaded with ICG

The following embolic properties are desirable: an initial injectable viscosity, a rapid transition to an occlusive gel after injection within the target vasculature, and the ability to achieve a modulus capable of resisting intraarterial pressures. To quantify these features, the viscoelastic properties were evaluated using rheology as previously described. Samples were analyzed using a temperature ramp from 18 to 37° C. (5.8° C./min) and a 20-mm, 4° cone geometry on a TA AR550-Stress Controlled Rheometer (New Castle, Del.). This was followed by a 3-hr. oscillatory time sweep at 37° C., 0.1% strain, and an angular frequency of 6.283 rad/s. Gelation and the potential for phase separation were evaluated using a tilt test. SELP 815K 12 wt % (400 μl) with 0.5 mg/mL of ICG was cooled in a glass chromatography vial (ThermoFisher Scientific, Waltham, Mass.) on ice. The vials were then hermetically sealed and placed into a 37° C. water bath in an upright position for 1, 2, 3, 5, 10, 15, 30, and 60 min. At each timepoint, each vial was briefly removed and photographed after being tilted 90°. The images were globally white-balanced and cropped to remove excess background.

v. ICG Release and Diffusion Behavior in Tissue Phantoms

Tissue-mimicking agar phantoms were used to measure ICG release from SELP and its diffusion behavior after simulating endovascular embolization. The agar phantoms were generated by dissolving 35 g/L of BD Bacto agar in deionized water prior to being autoclaved[22]. The solution was then cooled to <50° C., to which 200 mg/L of sodium azide and 35 g/L of BSA were added to respectively prevent bacterial growth and add a structural protein component. The phantom molds were cast in Cellstar® 6-well cell culture plates (Greiner, Austria) with segments of polyethylene (0.7-mm outer diameter) (Cole-Parmer, Vernon Hills, Ill.) that were threaded through pre-punched holes into each well. This process created a small void that ran through the center of each phantom to mimic the size of a blood vessel running through tissue that could be selectively embolized using clinical microcatheters. Each well was filled with 15 mL agar and allowed to gel at room temperature for 24 hrs. ICG was imaged within the phantoms using a 5-sec. exposure time and 780 nm excitation and an 831 nm emission with the Spectrum In Vivo Imaging System (IVIS) (Caliper Life Sciences, Massachusetts, USA). ICG (0.5 mg/mL) release from SELP and diffusion behavior was tested in triplicate for each phantom type. A non-SELP control, 0.5 mg/mL ICG in 50 mg/mL BSA in PBS, was tested to evaluate if SELP impaired the partitioning of ICG into the simulated tissue in each phantom type. ICG diffusion behavior was quantified by measuring its diffusional distances from the void over 48 hrs. A MATLAB script (The Mathworks, Inc., Natick, Mass.) was used to calculate the mean intensity of the fluorescent signal at varying distances from the center of the gel in images acquired on the IVIS. The radius at which the signal reached 10% of the maximum fluorescence intensity for each gel was designated as the visual front of diffusing dye.

vi. Correlation of ICG Imaging Between IVIS and a Clinical Endoscopic System

A Karl Storz 4 mm×18 cm ICG endoscope (Karl Storz, Tuttlingen, Germany) attached with a Power LED light source with fiber optic ICG cable and Karl Storz Image is Video System with ICG High Def Camera Head (Karl Storz, Tuttlingen, Germany) was used for imaging the ICG at different concentrations. ICG solution was prepared by mixing 25 mg of ICG in 1 mL of water, resulting in a 25 mg/mL solution. The solution was diluted by half in subsequent wells of a 96-well plate, and the last well was filled with only water (negative control). The endoscope's imaging head was held 4 mm above the surface of the 96-well plate to acquire images. Fluorescent light was captured by the camera and shown as blue. The intensity of the fluorescent light was quantified using the Zen Lite Blue software version 2.6 (Zeiss, Oberkochen, Germany). A fixed area of equal size for each well was selected, and the fluorescence was quantified by taking the arithmetic mean intensity of blue contribution for that particular region in pixels. IVIS was used to image ICG at different concentrations. Similar solutions of ICG were prepared and loaded in a 96-well plate. The same 96-well plate imaged with the Karl Storz endoscope was set on the sample stage. Living Image® Software (PerkinElmer, Massachusetts, USA) was used to take images of the 96-well plate. All the settings for acquiring the image were done in the software (Exposure time—1.50 sec.; Field of view—12.5 cm; F/stop(aperture)—2; Pixel Binning—medium). An overlay image combination of photographic and fluorescent image was recorded. The region of interest (ROI) tool was used to perform quantification of the surface intensities. Equal size ROIs were drawn on each well of the plate, and the radiant efficiency was recorded by the software for each defined ROI. The signal from the negative control well was subtracted to correct for background signal. The various radiant efficiencies were compared to different concentrations of ICG and results were plotted. Linear regression statistical analysis was also performed using GraphPad Prism 5 (GraphPad Software, San Diego, Calif.) to find correlations between the endoscopic fluorescent imaging and IVIS radiant efficiencies of ICG.

vii. Embolization of Microfluidic Models of Tumor Vasculature

Highly selective embolization requires the ability to pass through clinical microcatheters with small diameters and then selectively occlude target vasculature. To simulate tumor vasculature, a microfluidic device was designed based upon the Murray-Hess Law as previously described[16]. Devices representing tortuous conduits and of branching networks of tumor vasculature were constructed using Sylgard 184 silicone elastomer (Dow Corning, Midland, Mich.). Silicone was prepared per the manufacturer's instructions, de-aerated, poured over the mold, and cured at 153° C. After curing, the mold was removed, and the silicone was plasma-bonded to a glass microscope slide using an Enercon Dyne-A-Mite Air Plasma Surface Treater (Enercon, Menomonee Falls, Wis.). During testing, three microfluidic devices were connected in parallel to a central syringe pump. A 20-mL syringe was filled with PBS with red food color (McCormick & Company, Inc., Baltimore, Md.) for visualization. Saline was pumped through the devices at a flow rate of 0.63 mL/s to achieve a pressure of 40 mmHg, modeling rates and pressures found within blood vessels of similar cross-sectional area. SELP with 0.5 mg/mL ICG was injected into the microfluidic tumor model devices using 2.3-Fr, 110-cm microcatheter (Merit, South Jordan, Utah) submerged in a 37° C. water bath to simulate clinical procedures. The second and third devices, representing collateral vascular beds that feed nonmalignant tissue around the tumor, were connected in parallel to the test chip in order to evaluate the potential off-target embolization due to retrograde flow and provide an alternate path of flow after the occlusion of the tumor vasculature. After each test, the embolized device was replaced and the other chips investigated for evidence of occlusion using IVIS as described in the diffusion study. If no occlusion was observed, the collateral chips were reused. This experiment was replicated 3 times.

viii. Cytotoxicity of SELP ICG

L-929 and HUVEC were selected for use in assessing the cytotoxicity of the SELP-ICG embolic based upon their utility with respect to regulatory testing and relevance to the intended application of the device. L-929 cells are commonly used for FDA testing of contact cytotoxicity of medical devices, such as embolics. HUVEC represents a human cell line, another common cell that is also frequently used for evaluating cytotoxicity. Additionally, HUVEC represents vascular endothelial cells that embolic SELP will be in close contact with during in vivo administration. L-929 fibroblasts were grown with Dulbecco's Modified Eagle Medium (DMEM):F12 (1:1) media, supplemented with 10% FBS, and HUVECs were grown in ECGM. The cells were grown in T-75 flasks at 37° C. with 5% CO2 and passaged at 80-95% confluency. Cells were suspended using TrypLE™ Express Enzyme with no phenol red according to the manufactures protocol. The viability of cells was assessed using 0.4% trypan blue stain using a Countess Automated Cell Counter (ThermoFisher Scientific, Waltham, Mass.). L-929 cells were seeded into new T-75 flasks with 3×10⁵ to 6×10⁵ viable cells. HUVECs were seeded into new T-75 Flasks with 1×10⁵ to 7×10⁵ cells for each passage. Only cell cultures with greater than 90% viability, typically >95%, were used in assays. Cells were seeded into 96-well plates for testing before their 6th passage. SELP 815K 12% (wt/wt) with 0.5 mg/mL ICG and PBS with ICG 0.5 mg/mL were used as test samples and serially diluted to generate standard concentration curves. Viability was measured after 24 hrs using a Cell Counting Kit (CCK)-8 assay kit (Dojindo, Kumamoto, Japan). No treatment and 1% Triton-X were used as positive and negative controls, respectively. LD50 values were determined by fitting the data to a Hill plot with a variable slope using GraphPad Prism 5.0.

ix. Statistics

The data were collected and processed using Excel (Microsoft, Redmond, Wash.), and statistical and regression analyses were performed using GraphPad Prism 5.0. Outliers were identified using a Grubb's Test and excluded from cytotoxicity testing. The data analyzed in this study were assumed to be parametric in nature. Paired sets of data were analyzed using the Student's T-test for paired sets of data and one-way analysis of variance (ANOVA) with a post-hoc Bonferroni multiple comparison test to compare data sets with 3 or more groups. Least squares regression was used to assess linear correlations in the data. A p-value of less than 0.05 was used as the threshold for statistical significance.

3. Results

i. Effect of ICG on SELP Hydrogels

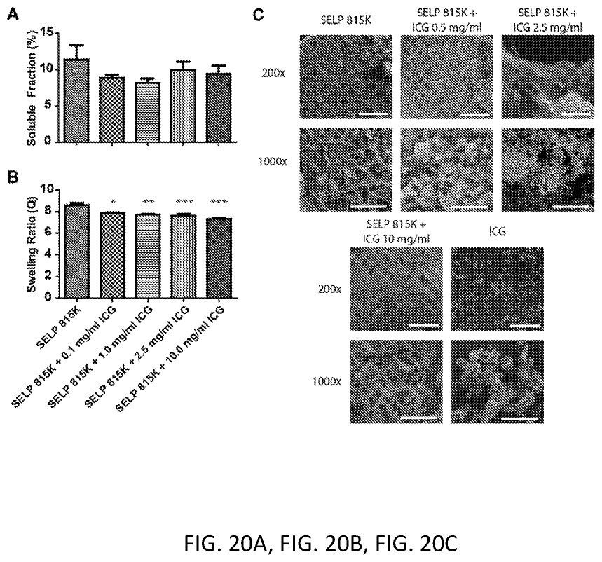

ICG increases SELP 815K polymer interactions, resulting in the formation of a denser hydrogel matrix. The soluble fraction of SELP is decreased by the addition of ICG (FIG. 20A). The swelling ratio of the SELP hydrogels significantly decreases with increasing concentrations of ICG. At 0.1 mg/mL ICG, the swelling ratio is decreased by 8.2% and at 10 mg/mL ICG, the swelling ratio decreased by 15.2% (FIG. 20B). While statistically significant, the relatively modest decrease in swelling ratio should not impact the gel's ability to occlude blood flow. Both of these trends indicate increased polymer-polymer interactions due to the presence of ICG. SEM imaging revealed visible changes in SELP microstructure after ICG incorporation (FIG. 20C). The matrix became appreciably denser with smaller voids and thicker partitions. Taken together, the addition of ICG to SELP tends to increase polymer interactions, leading to the formation of denser hydrogel matrices, which can alter viscosity and release kinetics.

ii. ICG Release from SELP

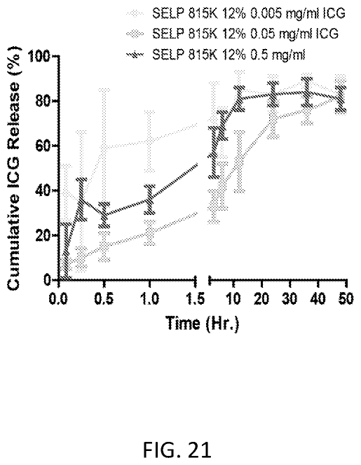

FIG. 20C demonstrates that ICG incorporation altered the microstructure of SELP in a concentration-dependent fashion. To investigate if these structural changes had an effect on ICG release, the release kinetics were evaluated by varying ICG-SELP compositions. ICG concentrations were selected to maximize the potential fluorescent signal in the context of tumor vasculature embolization and self-quenching behavior of ICG. The materials were injected through a 30 g needle and formed solid cohesive droplets at the bottom of the vials that did not phase separate. These features are necessary for the material to be able to be injected endovascularly, maintaining a high enough concentration to gel, and occlude the whole vascular lumen to prevent blood flow after administration. Burst release was greatest for 0.005 mg/mL group, which released 39±12% of the ICG payload within 5 min. of injection (FIG. 21). However, the relative burst release for higher concentrations of ICG was significantly reduced. The burst release was only 7±2% for the 0.05 mg/mL ICG group. The release profiles for 0.5, 0.05, and 0.005 mg/mL of ICG were consistent with first-order release kinetics and had n values ranging from 0.151±0.029 to 0.417±0.011 (mean±standard error) in the Korsmeyer-Peppas model, indicating that quasi-fickian diffusion was mediating the release of ICG. The 0.5, 0.05. and 0.005 mg/mL ICG gels released 84±6.0%, 72±8.0%, and 83±8.0%, respectively, of their payloads within 24 hrs., which is functional for pre-surgical embolization procedures as they are performed the day prior to tumor resection. Based upon these results and considering the anticipated volume of distribution of the dye during intravascular release within a tumor, SELP loaded with 0.5 mg/mL of ICG was selected for testing as an embolic material.

iii. Viscoelastic Properties of SELP Embolic Loaded with ICG

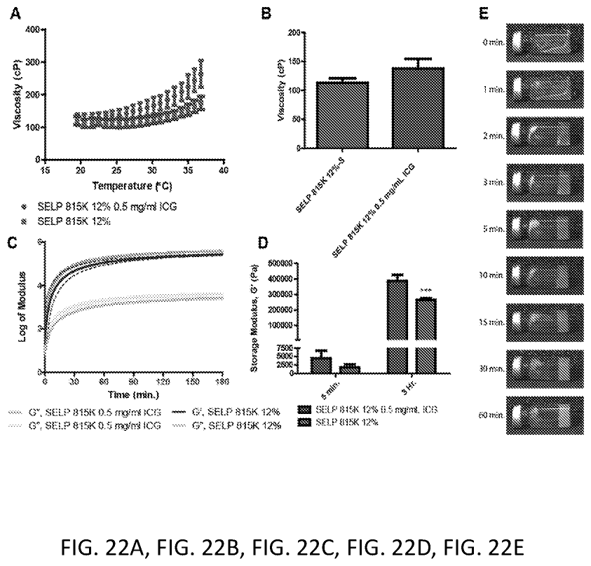

The incorporation of ICG increased the degree of the thermal viscoelastic response of SELPembolic solutions. Initially, the difference between SELP and SELP-ICG was negligible at 18° C. (120±13 cP and 123±17 cP, respectively). However, as the samples warmed, ICG incorporation accelerated the temperature-induced increase in SELP viscosity. At 37° C., the viscosity of SELP and SELP-ICG increased to 175±19 cP and 264±42 cP, respectively (FIG. 22A). This behavior indicates a 261% increase in the magnitude of the temperature-induced viscosity enhancement from 18 to 37° C. for SELP, due to ICG incorporation. However, below room temperature, the viscosities remain low enough for easy injection (FIG. 22B). ICG incorporation additionally increased the gelation kinetics and peak strength of the SELP embolic. The slope elevation of the storage modulus during an oscillatory time sweep at 37° C. indicates faster gelation (FIG. 22C). The 45.9% increase in SELP modulus due to ICG incorporation was highly significant (p<0.001, FIG. 22D). Within 2 min. at 37° C., SELP-ICG formed a network capable of resisting gravitationally-induced flow. A gradual increase in opacity with time indicates the continuing formation of microdomains within the SELP structure that scatter light and increasing the modulus of the material (FIG. 22E). No macroscopic phase separation was observed, indicating that the transition from an injectable liquid to an occlusive solid was isovolumetric, which is optimal for embolization.

iv. Release and Distribution of ICG in a Tissue Phantom

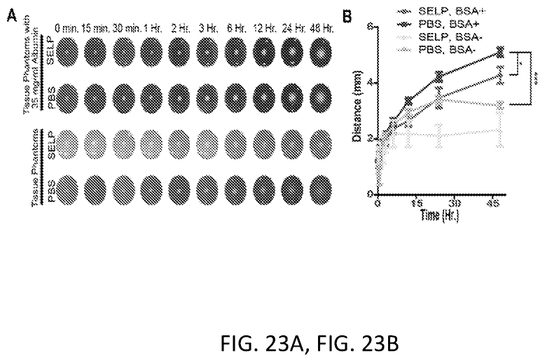

To fluorescently demarcate tumor margins, ICG must be released from SELP and diffuse into the tissues following embolization of the target vasculature. SELP-ICG was easily injected into tissue phantoms that simulated endovascular embolic delivery and subsequent release of ICG into the surrounding tissue. PBS was held within the channel by sealing the end prior to administration, which would correspond to embolization with a particle-based system immediately following ICG injection via a microcatheter. The incorporation of BSA into the phantom significantly increased both the relative intensity of the ICG and the distance by which ICG diffused within the phantom (FIG. 23A). Within 24 hrs. after SELP delivery, PBS control and ICG had diffused to a depth of 3.5±0.7 mm and 4.2±0.4 mm, respectively. In the phantoms without BSA, the ICG had only diffused to a depth of 2.1±0.6 mm, whereas PBS was measured at 3.4±0.4 mm. The difference between SELP with and without BSA is greater than that observed with PBS. Albumin enhanced diffusion and release of ICG from SELP within tissue phantoms. SELP reduced the distance ICG diffused likely by restricting the rate at which the dye was able to partition into the phantoms.

v. Imaging of ICG with Preclinical Tools Corelates with Clinically Available Endoscopics

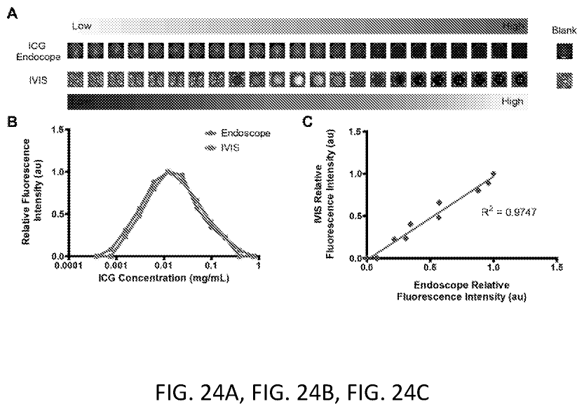

Imaging on IVIS correlates with imaging findings with a clinical endoscope system. The quenching effects of the ICG can clearly be seen in the images from both IVIS and the Karl Storz ICG endoscope (FIG. 24A). The optimal concentration of ICG was 0.012 mg/mL for both systems (FIG. 24B). Fluorescence intensity between the two systems was linearly correlated (FIG. 24C). The excitation light used by the endoscope gave the wells a green cast. At high concentrations of ICG, this light was absorbed by the ICG, resulting in darker wells. This confirms that IVIS is a capable tool for being able to assess the utility of ICG delivery systems for use with the Karl Storz ICG endoscope.

vi. Embolization in a Microfluidic Model of Tumor Vasculature

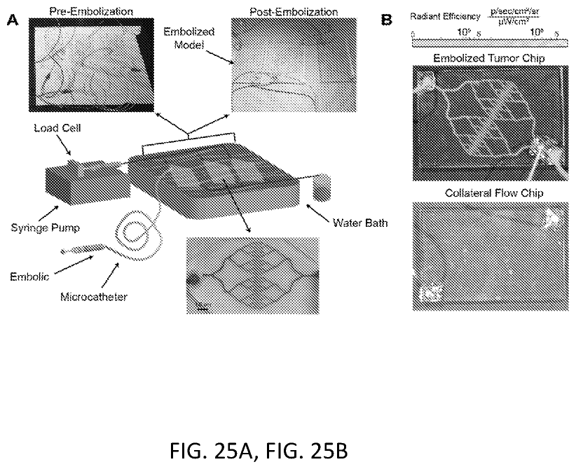

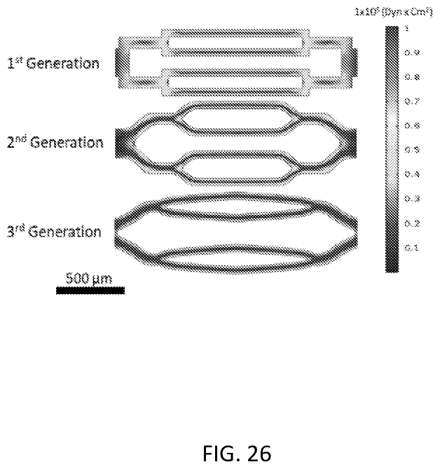

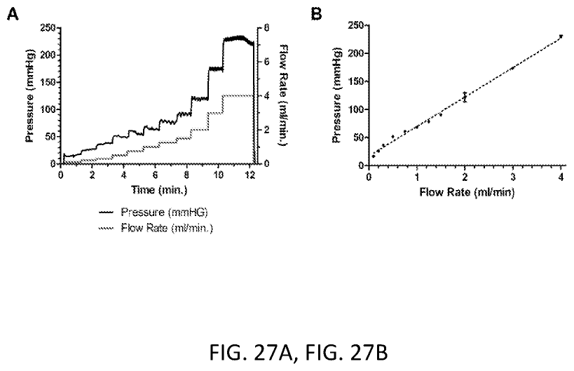

The ability to deliver locally via microcatheter and selectively occlude vasculature are essential features of embolic devices. The embolic capability of SELP-ICG was tested using custom-made, microfluidic tumor vasculature models with clinical microcatheters for simulating the anticipated implementation of the device (FIG. 25A). The microfluidic models went through several phases of design to minimize potential dead space (FIGS. 26 and 27). The flow-through resistance was 0.052 mmHg*min/L for 3 devices in parallel compared to 0.105 mmHg*min/L for a single vascular model. These resistances are not typical in human vascular beds, suggesting that the models herein are at least as challenging if not more so than in vivo vasculature of equivalent size. SELP-ICG was injected through a 2.3-Fr, 110-cm catheter submerged in a 37° C. water bath. SELP-ICG immediately occluded the vasculature upon reaching the device and redirected flow to the two collateral devices. Fluorescent imaging of the embolized device showed deep penetration and thorough occlusion of the entire device with no evidence of blockage or fluorescence in nontarget vascular models (FIG. 26B). These results were reproduced in three independent replications for the test embolization. SELP-ICG was able to effectively deliver ICG deep into the vasculature of a microfluidic model tumor, after successfully occluding flow.

vii. Cytoxicity of SELP ICG

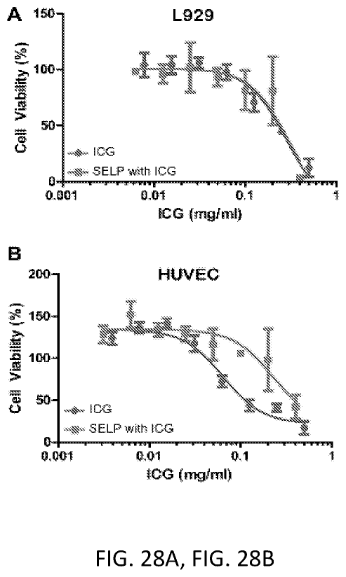

ICG cytotoxicity is ameliorated by SELP for HUVECs but not L929 fibroblasts. ICG is clinically used, but delivering a relatively high concentration locally can negatively impact cells. CCK-8 assay was used to assess the relative viability and health of the cells based on the reduction of a tetrazolium salt by dehydrogenase enzymes via electron mediators, such as nicotinamide adenine dinucleotide (NAD). The LD50 for L929 cells was not significantly different for ICG or ICG in SELP, 0.28±0.14 mg/mL and 0.30±0.11 mg/mL, respectively (FIG. 28A). However, the LD50 for ICG and ICG in SELP in HUVEC was very highly significant with respective values of 0.068±0.009 mg/mL and 0.23±0.03 mg/mL (FIG. 28B). In both cases, the addition of SELP either made no difference or ameliorated the toxic effects of ICG. This indicates that ICG incorporation into a hydrogel embolic is potentially feasible from a biocompatibility perspective.

4. Discussion

Achieving effective and efficient surgical resection of hypervascular tumors in the head and neck, such as JNAs, is challenging. Bleeding can rapidly obscure the endoscopic visual field, which increases the risk of surgical complications while reducing optimal surgical outcomes. The delineation of the boundary between malignant and healthy tissues can also be challenging, especially in the sinonasal cavity where margins are extremely difficult to obtain due to the proximity of critical anatomy within millimeters of the tumor. The development of an embolic system that delivers a tumor-selective dye can aid physicians by reducing intraoperative bleeding while demarcating tumor boundaries. While numerous embolic systems and strategies have been explored, no methods have reported the potential synergy between neoadjuvant tumor embolization and fluorescence-based image-guided surgery.

Effective drug delivery requires the precise delivery of the therapeutic agent with respect to location and time. In the context of fluorescence-based image-guided surgery, this means achieving high visual contrast by a localized dye within the tumor and minimizing dye diffusion to the surrounding healthy tissues. Due to its rapid clearance from the bloodstream (3-5 min. half-life), free ICG has a limited opportunity to accumulate in the tumor vasculature. Incorporation into nanoparticle formulations extends circulation time and can increase accumulation. However, bypassing the circulation phase of ICG accumulation entirely can elevate the local concentration of ICG beyond that which is achievable with freely circulating molecules.

The tunable biophysical properties of SELP embolics demonstrate promise as delivery vehicles for ICG to tumors. Release over an 18- to 24-hr. period is desirable for current neoadjuvant embolization practices for JNAs, which are typically embolized a day prior to surgery. Highly vascularized tumors have a reported average distance of 300-350 μm between blood vessels, and the vasculature occupies approximately 1% of the total tumor volume [26]. The 24-hr. diffusional distance of ICG released from SELP (FIG. 23) exceeds the intercapillary distances within tumors significantly and should fill the whole tumor volume. The delivery of 0.5 mg/mL of ICG also means that after 24 hrs. of release, the concentration of dye within the tumor will achieve the near-optimal concentration for maximizing fluorescent signal and avoid self-quenching effects (FIG. 24). This ability to concentrate at 24 hrs. is clinically relevant as JNA tumors are typically embolized 24 hrs prior to surgical resection.

The SELP-ICG embolic formulation has the potential to achieve distinct, fluorescently defined tumor margins that can be identified during fluorescence-based image-guided surgery. ICG has been shown in numerous human trials to preferentially accumulate within various types of solid tumors. These phenomena may be attributed to compromised lymphatic drainage in the malignant tissue, which in turn slows ICG clearance when compared to normal tissues. Delivering higher concentrations of ICG intratumorally via endovascular embolization can potentially further enhance accumulation, as the SELP embolic occludes tumor vessels and prevents ICG clearance by re-entry into tumor vessels. Clearance from the surrounding healthy tissue is unaffected and thus continues to produce a gradient of ICG at the tumor margin.

The incorporation of ICG into SELP increased gelation kinetics and stiffness of SELP embolics. ICG increased SELP intermolecular interactions as the material underwent phase transition. Polymer-polymer, polymer-solution, and polymer-solute interactions play a role in this behavior. The addition of ICG would increase the osmolarity of the solutions, which has previously been shown to accelerate network formation by increasing the relative favorability of polymer-polymer interactions. However, these observations alone do not explain the degree of enhancement seen in the viscosity profile. The divalent anionic character of ICG likely created bridging interactions between the positively charged lysine residues found within the elastinlike blocks of the SELP polymer backbone. At low temperatures, the polymers were sufficiently soluble that their Brownian motion rendered these interactions transient. As the temperature rose and the solubility of the polymers lessened, the relative strength of the ICG bridging-interaction increased, which led to the observed increase in viscosity.

Concentration, processing, local environment, and structure are the key features that contribute to the gelation and nano- and microscale formation behaviors of SELP. SELP penetrated into the venous outflow of the model while being rapidly and substantially diluted. The dilution prevented SELP from forming a cohesive network and the resulting soluble polymers were non-occlusive. SELP 815K, as was used in this study, does not gel below 2% (wt/wt) even after 24 hrs at 37° C. SELPs at concentrations below 2% form non-occlusive nanostructures ranging from fibers, spherical nanogels, globular single strand and proteins depending upon environmental conditions. SELP injection at 0.1 mL/min. into the simulated vascular beds, which were perfused at 38 mL/min., represents a 1/380 volumetric dilution. Therefore, the injection under the test conditions represents over a 60× safety margin. This property is similar to that of other clinically used embolic systems, such as LeGoo®, a poly(ethylene glycol)-polyethylene copolymer-based embolic gel, which passes into venous vasculature after producing a transient embolization.

The deliverability of SELP embolic was not compromised by the addition of ICG. The difference in viscosity was not significant between SELP and SELP-ICG at temperatures <25° C. Increased viscosity at 37° C. did not interfere with the ability of the SELP to perfuse into a clinically-relevant microfluidic model of tumor vasculature during simulated embolization (FIGS. 23 & 25). The SELP-ICG embolic was still also able to produce a thorough occlusion. Notably, the flow resistances in the microfluidic models were lower than that measured in tumor tissues, rendering the devices more difficult to embolize (FIG. 27).

Fluorescence visualized in tissue phantoms does not necessarily mirror what will be seen in a clinical setting (FIG. 23). Tumor tissues could require higher, or even lower, concentrations of dye to achieve an optimal fluorescent signal. It was also observed that albumin impacted both the release of ICG and the observed intensity of fluorescence (FIG. 23). ICG is known to interact with albumin and other globulins. The addition of albumin thus likely helps shield the negatively charged ICG from interacting with the positively charged lysines in the SELP backbone. Association with albumin also likely helps disperse ICG within the solution, which helps reduce ICG's self-quenching effects (FIG. 23 and FIG. 24). The auto-quenching effect of ICG can likewise complicate imaging as the intensity decreases, rather than increases, if the concentration of ICG is too high. This threshold appeared to be dependent upon factors, such as volume and geometry, which are difficult to control in a biological environment. As such, the optimal concentration of dye within the embolic cannot be determined without further in vivo testing.

The dual-functional embolization-visualization system, based upon SELP embolics and near IR dye ICG, was developed and characterized for future clinical application in combining embolotherapy with fluorescence-based image-guided surgery. Many of the strategies developed as part of this work could be additionally explored with clinically used materials. Similar to conventional trans-arterial chemoembolization procedures, ICG could be deployed intravascularly and immediately followed by embolization with a particle based-embolic. While this technique would allow for the assessment of ICG with embolotherapy using current clinical materials, the system developed herein offers potential clinical advantages over other materials. Controlled localized release of the fluorescent marker directly from the SELP embolic might enhance tumor demarcation contrast by increasing the intratumoral load, reducing intratumoral clearance, and reducing the amount of dye that enters healthy tissues.

5. Conclusions

ICG incorporation into and release from SELP embolic materials can be used. ICG-polymer interactions increase the viscosity, accelerate gelation, and increase the stiffness of the SELP embolics. ICG is deliverable from SELP over a clinically pertinent time frame. Combining embolization with delivery of a fluorescent dye to hypervascular tumors offers an opportunity to improve surgical visualization by reducing intraoperative bleeding, while simultaneously demarcating tumor margins.

### REFERENCES

[1] R. S. Bechan, M. E. Sprengers, C. B. Majoie, J. P. Peluso, M. Sluzewski, W. J. Van Rooij, Stent-assisted coil embolization of intracranial aneurysms: Complications in acutely ruptured versus unruptured aneurysms, Am. J. Neuroradiol. 37 (2016) 502-507. https://doi.org/10.3174/ajnr.A4542.
[2] B. G. Thompson, R. D. Brown, S. Amin-Hanjani, J. P. Broderick, K. M. Cockroft, E. S. Connolly, G. R. Duckwiler, C. C. Harris, V. J. Howard, S. C. C. la. Johnston, P. M. Meyers, A. Molyneux, C. S. Ogilvy, A. J. Ringer, J. Tomer, Guidelines for the Management of Patients With Unruptured Intracranial Aneurysms: A Guideline for Healthcare Professionals From the American Heart Association/American Stroke Association, 2015. https://doi.org/10.1161/STR.0000000000000070.
[3] J. W. Thompson, O. Elwardany, D. J. McCarthy, D. L. Sheinberg, C. M. Alvarez, A. Nada, B. M. Snelling, S. H. Chen, S. Sur, R. M. Starke, In vivo cerebral aneurysm models, Neurosurg. Focus. 47 (2019) 1-8. https://doi.org/10.3171/2019.4.FOCUS19219.
[4] N. Chalouhi, M. S. Ali, P. M. Jabbour, S. I. Tjoumakaris, L. F. Gonzalez, R. H. Rosenwasser, W. J. Koch, A. S. Dumont, Biology of intracranial aneurysms: Role of inflammation, J. Cereb. Blood Flow Metab. 32 (2012) 1659-1676. https://doi.org/10.1038/jcbfm.2012.84.
[5] D. J. Nieuwkamp, L. E. Setz, A. Algra, F. H. Linn, N. K. de Rooij, G. J. Rinkel, Changes in case fatality of aneurysmal subarachnoid haemorrhage over time, according to age, sex, and region: a meta-analysis, Lancet Neurol. 8 (2009) 635-642. https://doi.org/10.1016/S1474-4422(09)70126-7.
[6] E. S. Connolly, A. A. Rabinstein, J. R. Carhuapoma, C. P. Derdeyn, J. Dion, R. T. Higashida, B. L. Hoh, C. J. Kirkness, A. M. Naidech, C. S. Ogilvy, A. B. Patel, B. G. Thompson, P. Vespa, Guidelines for the management of aneurysmal subarachnoid hemorrhage: A guideline for healthcare professionals from the american heart association/american stroke association, Stroke. 43 (2012) 1711-1737. https://doi.org/10.1161/STR.0b013e3182587839.
[7] A. Adibi, A. Sen, A. P. Mitha, Cell Therapy for Intracranial Aneurysms: A Review, World Neurosurg. 86 (2016) 390-398. https://doi.org/10.1016/j.wneu.2015.10.082.
[8] I. Y. Tan, R. F. Agid, R. A. Willinsky, Recanalization rates after endovascular coil embolization in a cohort of matched ruptured and unruptured cerebral aneurysms., Interv. Neuroradiol. 17 (2011) 27-35. https://doi.org/10.1177/159101991101700106.
[9] Y. Niimi, J. Song, M. Madrid, A. Berenstein, Endosaccular treatment of intracranial aneurysms using matrix coils: Early experience and midterm follow-up, Stroke. 37 (2006) 1028-1032. https://doi.org/10.1161/01.STR.0000206459.73897.a3.
F. Briganti, G. Leone, M. Marseglia, G. Mariniello, F. Caranci, A. Brunetti, F. Maiuri, Endovascular treatment of cerebral aneurysms using flow-diverter devices: A systematic review, Neuroradiol. J. 28 (2015) 365-375. https://doi.org/10.1177/1971400915602803.
A. Poursaid, M. M. Jensen, E. Huo, H. Ghandehari, Polymeric materials for embolic and chemoembolic applications, J. Control. Release. 240 (2016) 414-433. https://doi.org/10.1016/j.jconrel.2016.02.033.
J. Hu, H. Albadawi, B. W. Chong, A. R. Deipolyi, R. A. Sheth, A. Khademhosseini, R. Oklu, Advances in Biomaterials and Technologies for Vascular Embolization, Adv. Mater. 31 (2019) 1-52. https://doi.org/10.1002/adma.201901071.
R. K. Avery, H. Albadawi, M. Akbari, Y. S. Zhang, M. J. Duggan, D. V. Sahani, B. D. Olsen, A. Khademhosseini, R. Oklu, An injectable shear-thinning biomaterial for endovascular embolization, Sci. Transl. Med. 8 (2016). https://doi.org/10.1126/scitranslmed.aah5533.
R. Price, A. Poursaid, J. Cappello, H. Ghandehari, Effect of shear on physicochemical properties of matrix metalloproteinase responsive silk-elastinlike hydrogels, J. Control. Release. 195 (2014) 92-98. https://doi.org/10.1016/j.jconrel.2014.07.044.
R. Dandu, A. Von Cresce, R. Briber, P. Dowell, J. Cappello, H. Ghandehari, Silk-elastinlike protein polymer hydrogels: Influence of monomer sequence on physicochemical properties, Polymer (Guildf). 50 (2009) 366-374. https://doi.org/10.1016/j.polymer.2008.11.047.
D. Hwang, V. Moolchandani, R. Dandu, M. Haider, J. Cappello, H. Ghandehari, Influence of polymer structure and biodegradation on DNA release from silk-elastinlike protein polymer hydrogels, Int. J. Pharm. 368 (2009) 215-219. https://doi.org/10.1016/j.ijpharm.2008.10.021.
R. Price, A. Poursaid, H. Ghandehari, Controlled release from recombinant polymers, J. Control. Release. 190 (2014) 304-313. https://doi.org/10.1016/j.jconrel.2014.06.016.
A. A. Dinerman, J. Cappello, H. Ghandehari, S. W. Hoag, Solute diffusion in genetically engineered silk-elastinlike protein polymer hydrogels, J. Control. Release. 82 (2002) 277-287. https://doi.org/10.1016/50168-3659(02)00134-7.
A. Poursaid, R. Price, A. Tiede, E. Olson, E. Huo, L. McGill, H. Ghandehari, J. Cappello, In situ gelling silk-elastinlike protein polymer for transarterial chemoembolization, Biomaterials. 57 (2015) 142-152. https://doi.org/10.1016/j.biomaterials.2015.04.015.
J. A. Gustafson, R. A. Price, K. Greish, J. Cappello, H. Ghandehari, Silk-elastin-like hydrogel improves the safety of adenovirus-mediated gene-directed enzyme-′prodrug therapy, Mol. Pharm. 7 (2010) 1050-1056. https://doi.org/10.1021/mp100161u.
R. Price, J. Gustafson, K. Greish, J. Cappello, L. McGill, H. Ghandehari, Comparison of silk-elastinlike protein polymer hydrogel and poloxamer in matrix-mediated gene delivery, Int. J. Pharm. 427 (2012) 97-104. https://doi.org/10.1016/j.ijpharm.2011.09.037.
J. A. Gustafson, R. A. Price, J. Frandsen, C. R. Henak, J. Cappello, H. Ghandehari, Synthesis and characterization of a matrix-metalloproteinase responsive silk-elastinlike protein polymer, Biomacromolecules. 14 (2013) 618-625. https://doi.org/10.1021/bm3013692.
J. Goode, Use of International Standard ISO 10993-1, “Biological evaluation of medical devices—Part 1: Evaluation and testing within a risk management process,” Dep. Heal. Hum. Serv. Food Drug Adm. (2016) 68. https://doi.org/http://www.fda.gov/downloads/medicaldevices/deviceregulationandguidance/guidancedocuments/ucm348890.pdf.
K. J. Isaacson, M. M. Jensen, D. B. Steinhauff, J. E. Kirklow, R. Mohammadpour, J. W. Grunberger, J. Cappello, H. Ghandehari, Location of Stimuli-Responsive Peptide Sequences within Silk-Elastinlike Protein-Based Polymers Affects Nanostructure Assembly and Drug-Polymer Interactions, J. Drug Target. 0 (2020) 1-47. https://doi.org/10.1080/1061186x.2020.1757099.
T. A. Altes, H. J. Cloft, J. G. Short, A. Degast, H. M. Do, G. A. Helm, D. F. Kallmes, Creation of saccular aneurysms in the rabbit: A model suitable for testing endovascular devices, Am. J. Roentgenol. 174 (2000) 349-354. https://doi.org/10.2214/ajr.174.2.1740349.
E. A. Ryan, L. F. Mockros, J. W. Weisel, L. Lorand, Structural origins of fibrin clot rheology, Biophys. J. 77 (1999) 2813-2826. https://doi.org/10.1016/S0006-3495(99)77113-4.
E. A. Ryan, L. F. Mockros, A. M. Stern, L. Lorand, Influence of a natural and a synthetic inhibitor of factor XIIIa on fibrin clot rheology, Biophys. J. 77 (1999) 2827-2836. https://doi.org/10.1016/S0006-3495(99)77114-6.
C. Yan, D. J. Pochan, Rheological properties of peptide-based hydrogels for biomedical and other applications, Chem. Soc. Rev. 39 (2010) 3528-3540. https://doi.org/10.1039/b919449p.
J. Gustafson, K. Greish, J. Frandsen, J. Cappello, H. Ghandehari, Silk-elastinlike recombinant polymers for gene therapy of head and neck cancer: From molecular definition to controlled gene expression, J. Control. Release. 140 (2009) 256-261. https://doi.org/10.1016/j.jconrel.2009.05.022.
J. L. Frandsen, H. Ghandehari, Recombinant protein-based polymers for advanced drug delivery, Chem. Soc. Rev. 41 (2012) 2696-2706. https://doi.org/10.1039/c2cs15303c.
M. H. Chen, Critical reviews in oral biology & medicine: Update on dental nanocomposites, J. Dent. Res. 89 (2010) 549-560. https://doi.org/10.1177/0022034510363765.
L. Feng, B. I. Suh, A. C. Shortall, Formation of gaps at the filler-resin interface induced by polymerization contraction stress. Gaps at the interface, Dent. Mater. 26 (2010) 719-729. https://doi.org/10.1016/j.dental.2010.03.004.
E. R. Stedronsky, J. Cappello, United States Patent (19) 11 U.S. Pat. No. 5,348,136, 1993.
U. Berlemann, O. Schwarzenbach, An injectable nucleus replacement as an adjunct to microdiscectomy: 2 year follow-up in a pilot clinical study, Eur. Spine J. 18 (2009) 1706-1712. https://doi.org/10.1007/s00586-009-1136-0.
K. J. Isaacson, M. M. Jensen, A. H. Watanabe, B. E. Green, M. A. Correa, J. Cappello, H. Ghandehari, Self-Assembly of Thermoresponsive Recombinant Silk-Elastinlike Nanogels, Macromol. Biosci. 18 (2018) 1-11. https://doi.org/10.1002/mabi.201700192.
J. N. Rodriguez, W. Hwang, J. Horn, T. L. Landsman, A. Boyle, M. A. Wierzbicki, S. M. Hasan, D. Follmer, J. Bryant, W. Small, D. J. Maitland, Design and biocompatibility of endovascular aneurysm filling devices, J. Biomed. Mater. Res.-Part A. 103 (2015) 1577-1594. https://doi.org/10.1002/jbm.a.35271.
S. O. Oktar, C. Yücel, D. Karaosmanoglu, K. Akkan, H. Ozdemir, N. Tokgoz, T. Tali, Blood-flow volume quantification in internal carotid and vertebral arteries: comparison of 3 different ultrasound techniques with phase-contrast MR imaging., AJNR. Am. J. Neuroradiol. 27 (2006) 363-9. http://www.ncbi.nlm.nih.gov/pubmed/16484412 (accessed May 7, 2018).
S. Juvela, Prevalence of and risk factors for intracranial aneurysms, Lancet. Neurol. 10 (2011) 595-597. doi:10.1016/S1474-4422(11)70125-9.
[1] Z. Liu, D. Wang, X. Sun, J. Wang, L. Hu, H. Li, P. Dai, The site of origin and expansive routes of juvenile nasopharyngeal angiofibroma (JNA), Int. J. Pediatr. Otorhinolaryngol. 75 (2011) 1088-1092. doi:10.1016/j.ijporl.2011.05.020.
[2] A. Christensen, K. Juhl, M. Persson, B. W. Charabi, J. Mortensen, K. Kiss, G. Lelkaitis, N. Rubek, C. von Buchwald, A. Kær, uPAR-targeted optical near-infrared (NIR) fluorescence imaging and PET for image-guided surgery in head and neck cancer: proof-of-concept in orthotopic xenograft model., Oncotarget. 8 (2017) 15407-15419. doi:10.18632/oncotarget.14282.
[3] P. S. Low, S. Singhal, M. Srinivasarao, Fluorescence-guided surgery of cancer: applications, tools and perspectives, Curr. Opin. Chem. Biol. 45 (2018) 64-72. doi:10.1016/J.CBPA.2018.03.002.
[4] N. Zhu, S. Mondal, S. Gao, S. Achilefua, V. Gruev, R. Liang, Dual-mode optical imaging system for fluorescence image-guided surgery., Opt. Lett. 39 (2014) 3830-2. doi:10.1364/OL.39.003830.
[5] E. J. Duffis, C. D. Gandhi, C. J. Prestigiacomo, T. Abruzzo, F. Albuquerque, K. R. Bulsara, C. P. Derdeyn, J. F. Fraser, J. A. Hirsch, M. S. Hussain, H. M. Do, M. V Jayaraman, P. M. Meyers, S. Narayanan, Society for Neurointerventional Surgery, Head, neck, and brain tumor embolization guidelines., J. Neurointerv. Surg. 4 (2012) 251-5. doi:10.1136/neurintsurg-2012-010350.
[6] R. Gupta, A. J. Thomas, M. Horowitz, Intracranial head and neck tumors: endovascular considerations, present and future, Neurosurgery. 59 (2006) S3-251-S3-260. doi:10.1227/01.NEU.0000239249.65742.1C.
[7] A. Poursaid, M. M. Jensen, E. Huo, H. Ghandehari, Polymeric materials for embolic and chemoembolic applications, J. Control. Release. 240 (2016). doi:10.1016/j.jconrel.2016.02.033.
[8] J. Mérian, J. Gravier, F. Navarro, I. Texier, Fluorescent nanoprobes dedicated to in vivo imaging: from preclinical validations to clinical translation, Molecules. 17 (2012) 5564-5591. doi:10.3390/molecules17055564.
[9] J. Hu, H. Albadawi, B. W. Chong, A. R. Deipolyi, R. A. Sheth, A. Khademhosseini, R. Oklu, Advances in biomaterials and technologies for vascular embolization, Adv. Mater. 31 (2019) 1901071. doi:10.1002/adma.201901071.
[10] J. Y. W. Chan, R. K. Y. Tsang, S. T. S. Wong, W. I. Wei, Indocyanine green fluorescence mapping of sentinel lymph node in patients with recurrent nasopharyngeal carcinoma after previous radiotherapy., Head Neck. 37 (2015) E169-73. doi:10.1002/hed.24052.
[11] H. Maeda, H. Nakamura, J. Fang, The EPR effect for macromolecular drug delivery to solid tumors: improvement of tumor uptake, lowering of systemic toxicity, and distinct tumor imaging in vivo, Adv. Drug Deliv. Rev. 65 (2013) 71-79. doi:10.1016/J.ADDR.2012.10.002.
[12] J. T. Alander, I. Kaartinen, A. Laakso, T. Panla, T. Spillmann, V. V. Tuchin, M. Venermo, P. Valisuo, A review of indocyanine green fluorescent imaging in surgery, Int. J. Biomed. Imaging. 2012 (2012) 1-26. doi:10.1155/2012/940585.
[13] R. P. Judy, J. J. Keating, E. M. DeJesus, J. X. Jiang, O. T. Okusanya, S. Nie, D. E. Holt, S. P. Arlauckas, P. S. Low, E. J. Delikatny, S. Singhal, Quantification of tumor fluorescence during intraoperative optical cancer imaging, Sci. Rep. 5 (2015) 16208. doi:10.1038/srep16208.
[14] R. Dandu, A. Von Cresce, R. Briber, P. Dowell, J. Cappello, H. Ghandehari, Silk-elastinlike protein polymer hydrogels: influence of monomer sequence on physicochemical properties, Polymer (Guildf). 50 (2009) 366-374. doi:10.1016/j.polymer.2008.11.047.
[15] W. Teng, J. Cappello, X. Wu, Recombinant silk-elastinlike protein polymer displays elasticity comparable to elastin, Biomacromolecules. 10 (2009) 3028-3036. doi:10.1021/bm900651 g.
[16] A. Poursaid, R. Price, A. Tiede, E. Olson, E. Huo, L. McGill, H. Ghandehari, J. Cappello, In situ gelling silk-elastinlike protein polymer for transarterial chemoembolization, Biomaterials. 57 (2015) 142-152. doi:10.1016/j.biomaterials.2015.04.015.
[17] A. Poursaid, M. M. Jensen, I. Nourbakhsh, M. Weisenberger, J. W. Hellgeth, S. Sampath, J. Cappello, H. Ghandehari, J. Cappello, Silk-elastinlike protein polymer liquid chemoembolic for localized release of doxorubicin and sorafenib, Mol. Pharm. 13 (2016) 2736-2748. doi:10.1021/acs.molpharmaceut.6b00325.
[18] J. A. Gustafson, R. A. Price, J. Frandsen, C. R. Henak, J. Cappello, H. Ghandehari, Synthesis and characterization of a matrix-metalloproteinase responsive silk-elastinlike protein polymer, Biomacromolecules. 14 (2013) 618-625. doi:10.1021/bm3013692.
[19] A. A. Dinerman, J. Cappello, H. Ghandehari, S. W. Hoag, Swelling behavior of a genetically engineered silk-elastinlike protein polymer hydrogel, Biomaterials. 23 (2002) 4203-4210.
[20] M. M. Jensen, W. Jia, K. J. Isaacson, A. Schults, J. Cappello, G. D. Prestwich, S. Oottamasathien, H. Ghandehari, Silk-elastinlike protein polymers enhance the efficacy of a therapeutic glycosaminoglycan for prophylactic treatment of radiation-induced proctitis, J. Control. Release. 263 (2017) 46-56. doi:10.1016/j.jconrel.2017.02.025.
[21] R. W. Korsmeyer, R. Gurny, E. Doelker, P. Buri, N. A. Peppas, Mechanisms of solute release from porous hydrophilic polymers, Int. J. Pharm. 15 (1983) 25-35. doi:10.1016/0378-5173(83)90064-9.
[22] Z. G. Portakal, S. Shermer, C. Jenkins, E. Spezi, T. Perrett, N. Tuncel, J. Phillips, Design and characterisation of tissue-mimicking gel phantoms for diffusion kurtosis imaging, Med. Phys. (2018). doi:10.1002/mp.12907.
[23] P. Gaehtgens, H. J. Meiselman, H. Wayland, Erythrocyte flow velocities in mesenteric microvessels of the cat, Microvasc. Res. 2 (1970) 151-162. doi:10.1016/0026-2862(70)90003-8.
[24] J. A. Carr, D. Franke, J. R. Caram, C. F. Perkinson, M. Saif, V. Askoxylakis, M. Datta, D. Fukumura, R. K. Jain, M. G. Bawendi, O. T. Bruns, Shortwave infrared fluorescence imaging with the clinically approved near-infrared dye indocyanine green, Proc. Natl. Acad. Sci. 115 (2018) 4465-4470. doi:10.1073/pnas.1718917115.
[25] H. Wang, X. Li, B. W.-C. Tse, H. Yang, C. A. Thorling, Y. Liu, M. Touraud, J. B. Chouane, X. Liu, M. S. Roberts, X. Liang, Indocyanine green-incorporating nanoparticles for cancer theranostics., Theranostics. 8 (2018) 1227-1242. doi:10.7150/thno.22872.
[26] H. K. Awwad, M. el Naggar, N. Mocktar, M. Barsoum, Intercapillary distance measurement as an indicator of hypoxia in carcinoma of the cervix uteri., Int. J. Radiat. Oncol. Biol. Phys. 12 (1986) 1329-33. doi:10.1016/0360-3016(86)90165-3.
[27] S. Luo, E. Zhang, Y. Su, T. Cheng, C. Shi, A review of NIR dyes in cancer targeting and imaging, Biomaterials. 32 (2011) 7127-7138. doi:10.1016/J.BIOMATERIALS.2011.06.024.
[28] M. Behbahaninia, N. L. Martirosyan, J. Georges, J. A. Udovich, M. Y. S. Kalani, B. G. Feuerstein, P. Nakaji, R. F. Spetzler, M. C. Preul, Intraoperative fluorescent imaging of intracranial tumors: a review, Clin. Neurol. Neurosurg. 115 (2013) 517-528. doi:10.1016/J.CLINEURO.2013.02.019.
[29] K. J. Isaacson, M. M. Jensen, A. H. Watanabe, B. E. Green, M. A. Correa, J. Cappello, H. Ghandehari, Self-assembly of thermoresponsive recombinant silk-elastinlike nanogels, Macromol. Biosci. 18 (2018) 1-11. doi:10.1002/mabi.201700192.
[30] J. Cappello, J. W. Crissman, M. Crissman, F. A. Ferrari, G. Textor, O. Wallis, J. R. Whitledge, X. Zhou, D. Burman, L. Aukerman, E. R. Stedronsky, In-situ self-assembling protein polymer gel systems for administration, delivery, and release of drugs, J. Control. Release. 53 (1998) 105-117. doi:10.1016/S0168-3659(97)00243-5.
[31] R. Price, A. Poursaid, J. Cappello, H. Ghandehari, Effect of shear on physicochemical properties of matrix metalloproteinase responsive silk-elastinlike hydrogels, J. Control. Release. 195 (2014) 92-98. doi:10.1016/j.jconrel.2014.07.044.
[32] R. Dandu, H. Ghandehari, J. Cappello, Characterization of structurally related adenovirus-laden silk-elastinlike hydrogels, J. Bioact. Compat. Polym. 23 (2008) 5-19. doi:10.1177/0883911507085278.
[33] S.-H. H. Jung, J.-W. W. Choi, C.-O. O. Yun, S. H. Kim, I. C. Kwon, H. Ghandehari, Direct observation of interactions of silk-elastinlike protein polymer with adenoviruses and elastase, Mol. Pharm. 12 (2015) 1673-1679. doi:10.1021/acs.molpharmaceut.5b00075.
[34] E. M. Sevick, R. K. Jain, Geometric resistance to blood flow in solid tumors perfused ex vivo: effects of tumor size and perfusion pressure., Cancer Res. 49 (1989) 3506-12. http://www.ncbi.nlm.nih.gov/pubmed/2731172 (accessed Jul. 2, 2019).
[35] T. Ishizawa, N. Fukushima, J. Shibahara, K. Masuda, S. Tamura, T. Aoki, K. Hasegawa, Y. Beck, M. Fukayama, N. Kokudo, Real-time identification of liver cancers by using indocyanine green fluorescent imaging, Cancer. 115 (2009) 2491-2504. doi:10.1002/cncr.24291.
[36] M. Varela, M. I. Real, M. Burrel, A. Forner, M. Sala, M. Brunet, C. Ayuso, L. Castells, X. Montañá, J. M. Llovet, Chemoembolization of hepatocellular carcinoma with drug eluting beads: efficacy and doxorubicin pharmacokinetics, J. Hepatol. 46 (2007) 474-481.

1. A method of treating an aneurysm in a subject comprising administering to the subject a therapeutically effective amount of a composition comprising a silk-elastinlike protein polymer (SELP).

2. The method of claim 1, wherein the aneurysm is a saccular aneurysm.

3. The method of claim 2, wherein the saccular aneurysm is a cerebral aneurysm (CA).

4. The method of claim 1, wherein the subject has been diagnosed with an aneurysm.

5. The method of claim 1, wherein the SELP embolizes the aneurysm.

6. The method of claim 1, wherein the SELP comprises the sequence of [GAGS(GAGAGS)ₙ₁(GVGVP)ₙ₂GXGVP(GVGVP)ₙ₃(GAGAGS)ₙ₄GA]ₙ₅GA, wherein X can be any amino acid, wherein n1 can be 2-10, wherein n2 can be 1-50, wherein n3 can be 1-50, wherein n4 can be 2-10, wherein n5 can be 1-14.

7. The method of claim 1, wherein the SELP comprises the sequence of [GAGS(GAGAGS)₂(GVGVP)₄GKGVP(GVGVP)₁₁(GAGAGS)₅GA]₆GA

8. The method of claim 1, wherein the SELP comprises the sequence of MDPVVLQRRDWENPGVTQLNRLAAHPPFASDPM[GAGS(GAGAGS)₂(GVGVP)₄G KGVP(GVGVP)₁₁(GAGAGS)₅GA]₆GAMDPGRYQDLRSHHHHHH

9. The method of claim 1, wherein the SELP transitions from a liquid to a hydrogel at temperatures above 23° C.

10. The method of claim 1, wherein the therapeutically effective amount is at least 1×, 2×, 3×, or 4× the aneurysm volume.

11. (canceled)

12. (canceled)

13. (canceled)

14. The method of claim 1, wherein the composition is administered using a catheter.

15. The method of claim 1, wherein the aneurysm comprises an aneurysmal sac, wherein the composition is administered into or enters the aneurysmal sac.

16. The method of claim 1, wherein no distal embolisms are present.

17. The method of claim 1, wherein the SELP remains in the aneurysm for one month.

18. The method of claim 1, wherein the composition further comprises a pharmaceutically acceptable carrier.

19. The method of claim 1, wherein the composition further comprises a contrast agent.

20. (canceled)

21. The method of claim 1, wherein the composition further comprises a visualization agent.

22. (canceled)

23. The method of claim 1, wherein the composition further comprises a therapeutic agent.

24. (canceled)

25. A method of preventing rupture of an aneurysm comprising administering to a subject having an aneurysm a composition comprising a SELP, wherein the SELP is present in the aneurysm and prevents rupture.

26.-48. (canceled)

49. A method of treating arterial venous malformations (AVM) in a subject comprising administering to the subject a composition comprising a SELP, wherein the SELP embolizes an abnormal blood vessel in the AVM.

50.-121. (canceled)
